(19) United States
(12) Patent Application Publication
(10) Pub. No.: US 2004/0220164 A1
(43) Pub. Date: Nov. 4, 2004

(54) Triaza compound immunoregulatory agents

(76) Inventors: Thomas Bell, Reno, NV (US); Dominique Schols, Herent (BE); Kaka Dey, Cohoes, NY (US); Kurt Vermeire, Tervuren (BE)

Correspondence Address: GREENLEE WINNER AND SULLIVAN P C, 5370 MANHATTAN CIRCLE, SUITE 201, BOULDER, CO, 80303, US

(21) Appl. No.: 10/680,076

(22) Filed: Oct. 6, 2003

Related U.S. Application Data
(63) Continuation of application No. PCT/US02/11223, filed on Apr. 8, 2002.
(60) Provisional application No. 60/282,212, filed on Apr. 6, 2001.

(51) Int. Cl. A61K 31/675

(52) U.S. Cl. 514/183; 514/79

(57) ABSTRACT

The invention provides certain macrocyclic triaza compounds which down-regulate CD4 expression for use in the treatment of autoimmune diseases and inflammatory diseases or conditions. In a specific embodiment, the invention provides certain naphthalene substituted triaza macrocycles which exhibit high activity for down regulation of CD4 expression. In particular, triaza macrocycles having dansyl groups are provided for use in pharmaceutical compositions.

### CROSS-REFERENCE TO RELATED APPLICATIONS

This application is a continuation of Patent Cooperation Treaty Application PCT/US02/11223, which designated the United States, filed Apr. 8, 2002 and takes priority under 35 U.S.C. 119(e) from U.S. provisional application serial No. 60/282,212 filed Apr. 6, 2001. Each of these patent applications is incorporated by reference in its entirety herein.

### FIELD OF THE INVENTION

This invention relates to the use of triaza compounds as immunomodulatory agents. More particularly, the present invention relates to the use of triaza compounds in downregulating the expression of CD4 on helper T-cells for the treatment of autoimmune disorders and inflammatory diseases including rheumatoid arthritis, psoriasis, insulin-dependent diabetes mellitus, systemic lupus erythematosus, inflammatory bowel diseases, multiple sclerosis, as well as non-autoimmune diseases including transplant rejection.

### BACKGROUND OF THE INVENTION

CD4 is a surface glycoprotein primarily expressed on the membrane of helper T-cells and monocytes, as well as some nonlymphocytic leukemic cell lines. CD4 serves a co-recognition function through interaction with MHC Class II molecules expressed in antigen presenting cells. CD4+ helper T-cells regulate T-cell and B-cell functions during T-dependent responses to viral, bacterial, fungal and parasitic infections.

During the pathogenesis of autoimmune diseases, CD4+ T-cells contribute to inflammatory responses which result in joint and tissue destruction. These processes are facilitated by the recruitment of inflammatory cells of the hematopoietic lineage, production of antibodies, inflammatory cytokines and mediators, and by the activation of killer cells. Rheumatoid arthritis (RA) is one manifestation of an autoimmune phenomenon which results in erosion, deformity, and destruction of joints. RA is characterized by elevated levels of activated CD4+ T lymphocytes in the affected joints. Currently there is no cure for RA. CD4+ cells have also been implicated in other chronic conditions including psoriasis, insulin-dependent diabetes mellitus, systemic lupus erythematosus, inflammatory bowel diseases, multiple sclerosis and other autoimmune diseases. Accordingly, it is desirable to down-regulate the autodestructive activity of CD4+ cells in cases of autoimmune disorders without compromising normal host defenses against opportunistic infections. Certain triaza compounds have been described in U.S. Pat. Nos. 5,663,161, 6,342,492 and U.S. application Ser. No. 09/769,021, publication no. 2002/0019423, published Feb. 14, 2002, as having anti-viral activities. Prior to the present invention, no immunomodulatory activities have been suggested for these compounds.

### SUMMARY OF THE INVENTION

The present invention provides methods of treating a subject suffering from a pathological condition wherein suppression of CD4+-T cell-mediated immune response is desirable by administering to the subject a therapeutically effective amount of a triaza compound. The present invention also provides pharmaceutical compositions for treatment of various autoimmune diseases and other disorders, other than the treatment of viral infection, wherein suppression of CD4+-T-cell mediated immune response is desirable. The present invention further provides pharmaceutical compositions for treatment of various chronic inflammatory conditions wherein suppression of CD4+-T-cell mediated immune response is desirable. These compositions comprise a therapeutically effective amount of one or more triaza compounds of this invention or a pharmaceutically acceptable salt or solvate thereof in combination with a pharmaceutically acceptable carrier appropriate for administration to an individual to be treated for the disease or disorder.

Pathological conditions which can be treated by practicing the present methods and employing the present compositions include autoimmune disorders and chronic inflammatory diseases, e.g., rheumatoid arthritis, type I-diabetes mellitus, autoimmune demyelinating diseases such as multiple sclerosis, inflammatory bowel disease syndrome, psoriasis, discoid lupus erythematosus, systemic lupus erythematosus (SLE), adult respiratory distress syndrome, cardiovascular atherosclerosis, and leukocytosis, as well non-autoimmune diseases such as graft-versus-host disease, transplant rejection, and asthma.

The present invention more specifically provides pharmaceutical compositions comprising a therapeutically effective amount of one or more a triaza compounds of this invention (or pharmaceutically acceptable salts or solvates thereof) in combination with a pharmaceutically accepted carrier for treatment of individuals having been diagnosed with or exhibiting symptoms of rheumatoid arthritis, type-I-diabetes, multiple sclerosis, psoriasis, lupus erythematosus and/or asthma. The invention also provides methods of treating these diseases, disorders or conditions by administering a pharmaceutical composition of this invention comprising a therapeutically effective amount of a triaza compound to an individual diagnosed with or exhibiting symptoms of any of rheumatoid arthritis, type-I-diabetes, multiple sclerosis, psoriasis, lupus erythematosus and/or asthma.

In another specific embodiment the present invention more specifically provides methods and pharmaceutical compositions for treating, minimizing or preventing transplant rejection. The method comprises administration of a therapeutically effective amount of one or more a triaza compounds of this invention (or pharmaceutically acceptable salts or solvates thereof) to an individual susceptible to transplant rejection.

Triaza compounds which can be employed in the present invention are represented by the following basic formula I:

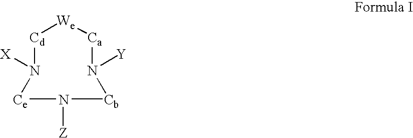

wherein:

W represents a bridge carbon which is unsubstituted, e.g., bonded to two hydrogens, or which is bonded directly or indirectly to one or two polar or non-polar side group substituents. Groups that can be bonded to the bridge carbon of W include those selected from the group consisting of alkyl groups (which together may form a cyclic group); double-bonded carbon (═C(H)₂ or ═C(R)₂), double bonded oxygen (═O), hydroxyl, alkyl of about one to 10 carbons (preferably of one to 6 carbon atoms) including, alkenyl of about two to 10 carbons (preferably of 2 to 6 carbon atoms); a substituted alkyl group carrying a polar group (e.g., a halide) or a charged substituent, such as an —S(R″)₂⁺, an —N(R″)₃⁺, a —PR₃⁺, or an —OSO₃⁻ group, alkoxy of about one to 10 carbons (preferably of one to 6 carbon atoms), aryl of about 6 to 12 carbons (preferably of about 7 to 10 carbons); halogen, methyl halogen(—CT₃,—CHT₂, or —CH₂T), methylene halide (═CT₂); optionally substituted epoxide (or oxirane); acyl (—CO—R); (—CO₂—R); CH₂OH and hydrogen; where halogen is F, Cl, I or Br; T, independently of other T, is F, Cl, I or Br, but preferably all T are the same halogen; R, independently of other R, is an optionally substituted alky of about one to 10 carbons (preferably of one to 6 carbon atoms), an optionally substituted alkenyl group of about 2 to 10 carbon atoms (preferably 2 to 6 carbon atoms)or an optionally substituted aryl group of about 6 to 12 carbons (preferably of about 7 to 10 carbons) and R″ is a hydrogen or an alkyl group having from one to 10 carbon atoms (preferably one to 6 carbon atoms); W may be bonded to one hydrogen and one polar or non-polar group;

X and Y independently represent an optionally substituted aryl group (Ar), an optionally substituted alkyl group having from one to 10 carbon atoms, or an optionally substituted alkenyl group having from 2 to 10 carbon atoms attached to the triaza macrocycle through an optional linker group L; where the linker group L can be sulfonyl (—SO₂—), —SO—, —PO—, —PO(OH)—, —PO(H)—, —PO₂(OH)—, —PO₂(H)—, —PO₃(OH)—, carboxy (—OCO—), carbonyl (—CO—), or alkyl (e.g., —(CH₂)ₙ— where n is 1 or 2; where Ar comprises at least one aromatic homocyclic or heterocyclic ring having from five to seven members; wherein the Ar ring can be substituted with one or more non-hydrogen substituent groups. Ar group substituents include one or more halogens, one or more —CN; one or more —SO₃, —SH, —SR or —S—OR groups; one or more trihalomethyl groups, e.g., —CF₃; one or more NO, one or more NO₂, one or more NH₂, NHR or N(R)₂ groups; one or more alkyl groups, one or more alkoxy groups, one or more hydroxyl groups, one or more acyl groups (—COH or —CO—R), one or more acid or ester groups (—CO₂H or —CO₂R, respectively), where R, independently of other R, is an alky of about one to 10 carbons or an aryl group of about 7 to 10 carbons (preferably of about 7 to 10 carbons) and where X and Y are not both an alkyl group;

Z represents a hydrogen, or optionally substituted aryl, alkyl or alkenyl groups attached to the triaza macrocycle though a linking group L³, wherein the aryl, alkyl and alkenyl groups and the linking group of Z are as described under X and Y variables above;

C labeled with subscripts a-d in formula I represent carbon bridges, preferably alkylene bridges, between nitrogens, these carbon bridges, the length of which is defined by the values of subscripts a-d and e, may all be the same length or may differ in length, each bridges may be composed entirely of saturated alkyl groups (e.g. —(CH₂)ₐ—), or one or more bridges may contain one or more double or triple bonds between carbons, additionally one or more bridge carbons can be optionally substituted with one or more polar groups, for example, halogens or hydroxy groups, and additionally aromatic (including heteroaromatic), non-aromatic (cycloalkyl or cycloalkenyl) rings or both may be fused to one or more of the carbon atom bridges; and

a and d, independently, represent a number from zero to 10; b and c, independently, represent a number from one to 10; and e represents a number from zero to three; and preferably, a+d+e≧1; and the formula contains sufficient hydrogens for a stable molecule.

Compounds useful in the pharmaceutical compositions of this invention and methods of this invention include pharmaceutically acceptable salts and solvates of the compounds of formula I as well as those of triaza compounds of other formulas listed below.

Compounds useful in the pharmaceutical compositions of this invention and in the methods of this invention include pharmaceutically acceptable prodrugs which after administration provideddesired active triaza compounds of this invention in vivo.

In pharmaceutically acceptable salts of the compounds of formula I, one or more of the N of the macrocyclic ring can be protonated and the salt then contains an appropriate number of pharmaceutically acceptable anions. Pharmaceutically acceptable anions include, among others, halide (generally), chloride, bromide, and iodide (more specifically), sulfates, bisulfates, phosphates, anions of organic mono and diacids (generally), acetate, maleate, fumarate, oxalate, lactate, tartrate, citrate, gluconate, methanesulfonate, thionates (generally), isethionate (HOCH₂CH₂SO₃⁻), salicylate, and 4-toluene-sulfonate. Compounds of this invention may be solvates, particularly hydrates.

W groups include, among others, >C═CH₂; >C═CT₂ (where T is a halide); >C═CHT (where T is a halide); .>CTR or >CHR (where T is a halide and R is an optionally substituted alkyl or aryl group); >CRR (where R is an alkyl, alkenyl or aryl group, each R may be same or different, each R is optionally substituted and two R's together can form a cyclic group (e.g., a cycloalkyl group or a cyclic ether group). Other W groups are illustrated in the chemical formulas herein. Preferably, W is ethene (>C═CH₂), ethylene dihalide (>C═CT₂) or the bridge carbon of W is bonded to H and —CH₂—OH, X and Y are L—Ar groups where L is sulfonyl and Ar is an optionally substituted phenyl or naphthalene, Z is an optionally substituted alkyl group having 5 or more carbons, or an L³—Z′ group where L is carboxy, carbonyl or alkyl linker and Z′ is an optionally substituted phenyl, cyclohexyl or cyclohexenyl group, a and d are one or two, e is one, and b and c are 2-4.

Preferably at least one of X or Y comprises an aromatic group.

More preferably, W is ethene, X and Y are independently selected from tosyl or dansyl groups or both, Z is benzyl, —CH₂-cyclohexyl or —CH₂-cyclohexenyl, a, d, and e are one, and b and c are three. More preferred triaza compounds of this invention include CADA, KKD015, KKD016, QJ023, QJ028, QJ036, QJ037, QJ038, QJ033, HJC321, A8117, and AS-PB127 and pharmaceutically acceptable salts or solvates thereof Most preferred compounds for use in the present methods are cyclotriazadisulfonamide (CADA), KKD015 and KKD016 and pharmaceutically acceptable salts or solvates thereof.

Preferred triaza compounds for use in the methods herein include those exhibiting IC₅₀ (CD4) less than about 10 μg/ml as measured by any method described herein or known in the art in any appropriate cell type, e.g., MT4 cells, or SupT1 cells. More preferred compounds are those exhibiting IC₅₀ (CD4) below about 5 μg/ml. Most preferred triaza compounds for use in the methods herein include those exhibiting IC₅₀ (CD4) less than about 3 μg/ml.

In a specific preferred embodiment, the invention provides triaza compounds which exhibit unexpectedly high activity for suppression of CD4 expression. These high activity triaza compounds comprise one or two optionally substituted naphthalene groups linked to the triaza macrocycle through a sulfonyl linker, and particularly include dansyl groups or analogues thereof. In these high activity triaza compounds the dansyl group or analogues thereof is a fluorophore that can be useful in tracing the triaza compound itself or in experiments assessing the mechanism(s) of down-regulation of CD4 expression. The invention also provides methods of treatment of autoimmune diseases, conditions or disorders and treatment of inflammatory diseases, conditions or disorders employing theses highly active triaza compounds with naphthalene substituents.

### BRIEF DESCRIPTION OF DRAWINGS

FIG. 1. Structure of cyclotriazadisulfonamide (CADA). Skeletal diagram of the hydrochloride salt (C₃₂H₄₀N₃C1S₂0₄, MW 618).

FIGS. 2A-2C. Surface CD4 expression in MT-4 cell line, SupT1 cell line and PBMCs after incubation with CADA hydrochloride (3.2 or 16 μM) for 1 and 4 days. (A) Cell surface CD4 expression of control MT-4 cells and MT-4 cells treated with CADA (3.2 μM) for 1 day or 4 days. (B) Cell surface CD4 expression of control SupT1 cells and SupT1 cells treated with CADA (3.2 μM) for 1 day or 4 days. (C) Cell surface CD4 expression of control PBMCs and PBMCs treated with CADA (16 μM) for 1 day or 4 days. The cells were stained with anti-CD4 mAb (Leu3a-PE). Percentages of fluorescent cells and MFI (mean fluorescence intensity) are indicated in each histogram.

FIGS. 3A-3B. Intracellular CD4 staining in SupT1 cells after incubation with CADA hydrochloride (3.2 μM) for 4 days. (A) The intracellular CD4 expression of SupT1 cells cultured in medium alone and (B) treated with CADA for 4 days.

FIG. 4. Comparison of the effects on CD4 modulation by ATA (24 μM), CADA hydrochloride (16 μM) and PMA (8 nM) in SupT1 cells after incubation with the compounds analyzed after 20 minutes, 4 hours, 1 day and 4 days.

FIG. 5A-D. Correlation between anti-HIV potency and CD4 down-modulating capability of CADA (5A), QJ023(5B), QJ028 (5C) and QJ033 (5D). MT-4 cells were infected with NL4.3 in the presence of different doses of the compounds. After 4 days, supernatant was collected and analyzed for its p24 content (vertical bars). In parallel, uninfected MT-4 cells were treated with the same doses of the analogs, and CD4 expression was analyzed flow cytometrically after 4 days of incubation (line). The MFI of the Leu3a-FITC staining is calculated for the different doses of CADA, QJ023, QJ028 and QJ033, and is expressed as percentage of the MFI of control MT-4 cells.

FIG. 6. Correlation of the anti-HIV-1 (NL4.3) activity and CD4 expression down-modulation of the different CADA analogs (i.e. CADA, QJ023, QJ027, QJ028, QJ029, QJ030, QJ033, QJ035, QJ036, QJ037, QJ038, QJ040, QJ041, AS-N6P6, 95-213, 98-035, HJC321, AS117 and AS-PB127) in MT-4 cells as assessed by linear regression analysis. For each analog, the anti-HIV-1 activity (IC₅₀ value in μg/ml) is plotted against the CD4 down-modulating capability (IC₅₀ value in μg/ml calculated from the mean fluorescence intensity of MT-4 cells labeled with the FITC-conjugated anti-CD4 mAb).

FIG. 7. Correlation of the anti-HHV-7 activity and CD4 expression down-modulation of the different CADA analogs (i.e. CADA, QJ023, QJ027, QJ028, QJ029, QJ033, QJ036, QJ037, QJ038, QJ041, AS-N6P6, HJC321, AS117, AS-PB127 and MFS-SC001) in SupT1 cells as assessed by linear regression analysis. For each analog, the anti-HHV-7 activity (IC₅₀ value in μg/ml) is plotted against the CD4 down-modulating capability (IC₅₀ value in μg/ml calculated from the mean fluorescence intensity of SupT1 cells labeled with the FITC-conjugated anti-CD4 mAb).

### DETAILED DESCRIPTION OF THE INVENTION

The present inventors have surprisingly found that a family of synthetic triaza compounds are capable of down-regulating the level of CD4 expression on the surface of T-cells. Accordingly, the present invention is directed to the use of synthetic triaza compounds as immunomodulatory agents in down-regulating the CD4 expression. Most generally, the triaza compounds useful in this invention are represented by chemical formula I and include pharmaceutically acceptable salts and solvents thereof.

The following terms are defined:

The term “alkyl” is used generally as it is understood in the art and is intended to encompass straight-chain saturated hydrocarbon, branched chain saturated hydrocarbons as well as cyclic hydrocarbons. Exemplary alkyl groups are methyl, ethyl, propyl, isopropyl, butyl, sec-butyl, tertiary butyl, pentyl, isopentyl, neopentyl, tertiary pentyl, 1-methylbutyl, 2-methylbutyl, 3-methylbutyl, hexyl, isohexyl, heptyl, octyl, cyclopropyl, cyclobutyl, cyclopentyl and cyclohexyl groups. Alkyl groups can have portions that are cyclic combined with straight-chain or branched portions, such as methylcyclohexane. Alkyl groups herein can be optionally substituted particularly with one or more halogens, one or more hydroxyls or with one or more other polar groups which may, for example, enhance compound solubility in water or aqueous solutions.

The term “alkenyl” is used generally as it is understood in the art and is intended to encompass unsaturated hydrocarbons having one, two or more double bonds, preferred alkenyl groups will have one or two double bonds. Alkenyl groups may have portions that are straight-chain, branched and/or cyclic. Exemplary alkenyl groups are ethylene, propylene, butylene, butadiene, pentylene, pentadiene, hexylene, hexadiene, heptylene, heptadiene, octylene, octadiene, cyclopentylene, cyclopentadiene, cyclohexylene, cycloheptylene, cyclooctylene, cyclooctadiene and various isomers thereof. Alkenyl groups herein can be optionally substituted particularly with one or more halogens, one or more hydroxyls or with one or more other polar groups which may, for example, enhance compound solubility in water or aqueous solutions.

The term “alkoxy is used generally as it is understood in the art and is intended to encompass alkyl groups as defined above bonded through an oxygen (—OR, where R is an alkyl group). Exemplary alkoxy groups are methoxy, ethoxy, propoxy, isopropoxy, butoxy, isobutoxy, tertiary butoxy, pentoxy, isopentoxy, neopentoxy, tertiary pentoxy, hexoxy, isohexoxy, heptoxy and octoxy. The alkyl portion of the alkoxy group can be optionally substituted as indicated above for alkyl groups.

The term ether is used generally as it is used in the art to refer to hydrocarbons containing one or more C—O—C linkages which may be between two alkyl and/or alkenyl and/or aryl groups. Exemplary ether groups include among others: —(CH₂)ₐ—O-alkyl, —(CH₂)ₐ—O-alkenyl, —(CH₂)ₐ—O-aryl, —(CH₂)a—O—(CH₂)_{b}—O-alkyl, —(CH₂)ₐ—O—(CH₂)_{b}—O-alkenyl, —(CH₂) ₐ—O-(CH₂)_{b}—O-aryl, —(CH₂)ₐ—O—(CH₂)_{b}—O—(CH₂)_{c}—O-alkyl, etc., and where a, b and c are integers that may be the same or different and can for example range from 1 to 6. The ether linkage may also be within a saturated or unsaturated ring, e.g.,:

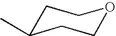

The term thioether is defined analogously to ether wherein the term refers to hydrocarbons as illustrated for ethers but which contain one or more C-S-C linkages.

The term “aryl” is used generally as it is used in the art and is intended to encompass groups or moieties which comprise at least one aromatic ring. The ring may be an aromatic ring of any size but is typically a ring of 5 or 6 atoms which may all be carbon or which may contain one, two or three heteroatoms. The term “aromatic” is used as it is generally used in the art to refer to rings having a conjugated π-electron system. The term aryl encompasses both homocyclic (carbocyclic) aromatic rings (e.g., phenyl rings, naphthalene rings) and heteroaromatic rings (e.g., pyridine rings). The term encompasses groups or moieties containing two or more rings (again preferably 5 or 6 member rings) which may be fused rings wherein at least one of the rings is aromatic. Aryl rings can be optionally substituted. Exemplary aryl rings include phenyl, naphthalene, biphenyl, pyridine, pyrimidine, thiophene, etc. Exemplary fused rings include naphthalene, phenanthrene, anthracene, indole, quinoline, isoquinoline, carbazole, benzimidazole and benzofuran.

The term “optionally substituted” most generally is used to refer to possible substitution of one or more substituents on alkyl, alkenyl and/or aryl groups each of which may be straight-chain, branched or cyclic. Most generally the term does not indicate the site of substitution. However, the site of substitution may be indicated by chemical nomenclature or in illustrative chemical structures, e.g., para-substitution on phenyl rings. In certain cases, the point of substitution of a given substituent on a given alkyl, alkenyl or aryl group will be clear to one of ordinary skill in the art. Optionally substitution refers to substitution by one or more charged, polar or non-polar groups. Charged substituents, for example, an —S(R″)₂⁺, an —N(R″)₃⁺, a —PR₃⁺, or an —OSO₃⁻ group among others, where R″ is independent of other R″ a hydrogen or an alkyl group having from one to 10 carbon atoms (preferably one to 6 carbon atoms), can enhance solubility of the triaza compounds in water or aqueous solutions or can be employed to generate prodrugs which are converted into desired active triaza compounds in vivo. Polar substituents, for example, include halogen, —CT₃, —NH₂, —N(R)₂, —NO, —NO₂, —SH, —SO₃H, —SO₃R, —OH, —COH, —COR, —CONH—, ——CONR—, —CO₂H, and —CO₂R (where each T independently is a halogen and where each R independently is alkyl or alkenyl having one to 10 carbon atoms, and preferably one to 6 carbon atoms, or aryl). These polar groups are capable of aiding solubility of the compounds. Nonpolar or less polar substituent groups include alkyl groups, alkenyl groups and unsubstituted aryl groups as well as —OR, —(CH₂)n—OR (n is an integer 1 or more, ethers), —SR, —(CH₂)ₙ—SR (n is an integer 1 or more, thioether). In general, ether and thioether substituent groups can contain more than one oxygen or sulfur atoms, respectively. Alkyl, alkenyl or aryl R groups of substituents can themselves be substituted with one or more polar or non-polar groups, e.g., halogens or hydroxy, such substituents include for example halogenated alkyl groups and hydroxylated alkyl groups.

The term “pharmaceutically acceptable” as applied to carriers, diluents, excipients, prodrugs, salts and/or other ingredients of pharmaceutical compositions means that the component must be compatible with the other ingredients of the formulation, and not unduly deleterious to the human or animal patient treated with the composition. The pharmaceutically acceptable ingredients generally do not exhibit undue toxicity, irritation, allergic response, and the like, commensurate with a reasonable benefit/risk ratio as appreciated by those in the art.

A “prodrug” refers to a compound that is a drug precursor which releases a selected drug in vivo after administration to an individual. The compound is typically related in structure to the drug and may have a particular advantage for formulation (e.g., increased stability) or administration (e.g., increased solubility). After administration, a chemical or physiological process (e.g., a change in pH or enzyme action) converts the prodrug to the desired drug. Prodrugs of the triaza compounds herein are intended to be encompassed herein. Those of ordinary skill in the art can, in view of what is generally known in the about prodrug structure and the chemical and enzymatic process that can occur on administration can devise prodrug of triaza compounds of this invention. Triaza compounds of this invention carrying charged substituents, such as one or more —S(R″)₂⁺, —N(R″)₃⁺, —PR₃⁺, —OSO₃⁻ groups or combinations thereof, where R″, independent of other R″, is a hydrogen or an alkyl group having from one to 10 carbon atoms can function as prodrugs.

In one embodiment, the present invention provides methods of treating an individual suffering a pathological condition wherein suppression of CD4+-T cell-mediated immune response is desirable by administering to the subject a therapeutically effective amount of a triaza compound. The triaza compound or its pharmaceutically acceptable salt or solvate is typically administered as a pharmaceutical composition which comprises one or more triaza compounds present in an amount or a combined amount sufficient to provide a therapeutic effect.

The term “individual” as used herein is meant to include all mammalian subjects, e.g., humans and primates. Typically the individual to be treated has been diagnosed with or is exhibiting the symptoms of the disorder, disease or condition to be treated.

By “treating” a pathological condition is meant the symptoms of the pathological condition are ameliorated. The term “treating” as used herein also encompasses delaying or preventing the onset of a pathological condition.

According to the present invention, pathological conditions which can be treated by practicing the instant methods include autoimmune disorders and chronic inflammatory diseases wherein suppression of CD4+-T cell-mediated immune response is desirable, e.g., rheumatoid arthritis, type I-diabetes mellitus, multiple sclerosis, inflammatory bowel disease syndrome, psoriasis, discoid lupus erythematosus, systemic lupus erythematosus (SLE), adult respiratory distress syndrome, cardiovascular atherosclerosis, and leukocytosis. Other pathological conditions which can also be treated by practicing the instant methods include non-autoimmune diseases such as graft-versus-host disease, transplant rejection, and asthma. The term “disease-associated CD4+-T cell-mediated immune response” is defined herein to refer to all CD4+-T cell-mediated immune responses in an animal (including any mammal, as well as humans) that result in or contribute to a pathological condition, disorder or disease. The term refers particularly to those CD4+-T cell-mediated immune responses the suppression of which is associable to an improvement in the pathological condition.

Triaza compounds which can be used in the present methods to down-regulate the CD4 expression are described in U.S. Pat. No. 5,663,161, the entirety of which is incorporated herein by reference. These triaza compounds are represented by the basic formula I:

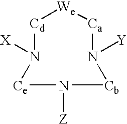

wherein the variables, X, Y, Z, W, a, b, c, d and e have been defined above in the Summary of the Invention.

The compounds are characterized as having at least three nitrogen atoms (amine sites) linked by at least three alkylene bridges to form a triazamacrocycle ring containing the nitrogens. The alkylene bridge linking groups are preferably alkanes containing from one to 10 carbons, but more preferably the alkylene bridges have 2, 3 or 4 carbons. The alkylene bridges linking the nitrogen atoms can additionally contain one or more double or triple bonds (typically one in preferred length bridges) and/or include aromatic, non-aromatic rings or both fused to the alkylene bridge. Bridges containing fused rings and linking two nitrogens of the triamine structure are exemplified by the following:

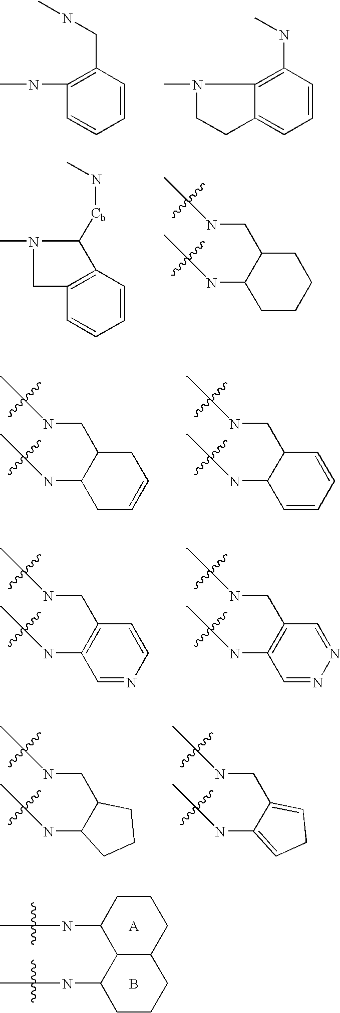

and isomers of these structures, where all rings indicated can be optionally substituted and the A and B rings can be optionally substituted cycloalkyl, cycloalkenyl and/or aryl groups, e.g., the fused A-B ring can be optionally substituted naphthalene.

In the above formula, c and b are preferably 2-4 and more preferably are both 3. The alkylene bridges are preferably saturated, unsubstituted and do not have fused rings attached thereto, e.g., —(CH₂)₃—. In the above formula, a, d and e are preferably 1 or 2 and more preferably are each 1.

The bridge carbon (designated W) of the third alkylene bridge may be double-bonded to another carbon which is in turn bonded to one or two R groups (i.e., >C═C(R)₂ or bonded to or functionalized with at least one substituent which is a polar group.

Representative groups for W include:

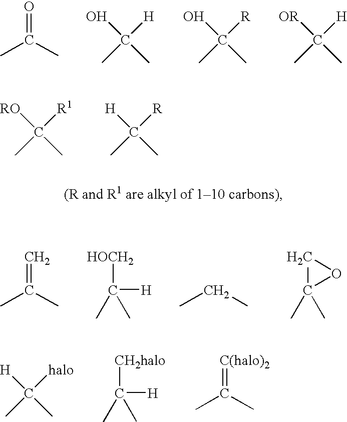

where halo is F, Cl, Br, or I. W may also be unfunctionalized (i.e., bonded to two hydrogens). In preferred embodiments W is >C═CH₂ or >C═C(halogen)₂.

In preferred embodiments X and Y are optionally substituted aromatic groups linked through L groups to the nitrogen macrocycle. These aromatic groups can contain nitrogen, sulfur or oxygen heteroatoms and preferably comprise one or more 5- or 6-member aromatic rings. The aromatic groups may contain a single aromatic ring or two or more fused rings at least one of which is an aromatic ring. Exemplary aromatic rings include phenyl, pyrrolyl, furanyl, thiophenyl, pyridiyl, thiazoyl, etc. Aromatic rings can be substituted for example with hydrophilic or polar groups.

The alkyl or alkenyl groups for X and Y may be branched or unbranched and include up to ten carbons. Typical examples of alkyl and alkenyl groups for X and Y include methyl, ethyl, n-propyl, isopropyl, n-butyl, sec-butyl, tert-butyl, isobutyl, pentyl, hexyl, heptyl, octyl, nonyl, decyl, ethenyl, propenyl, butenyl, pentenyl, hexenyl, heptenyl, octenyl, nonenly, decenyl, butadienyl, pentadienyl, hexadienyl, heptadienyl, octadienyl, nonadienyl, decadienyl. The alkyl or alkenyl groups may be in whole or in part in the form of rings such as cyclopentyl, cyclopentenyl, cyclopentadienyl, cyclohexyl, cyclohexenyl, cyclohexadienyl, cycloheptyl, cycloheptenyl and cyclohexylmethyl. The cyclic groups may be further substituted with alkyl or aryl groups. Preferred alkyl and alkenyl groups in substituents contain from one to six carbon atoms and more preferably contain one to 3 carbon atoms.

Preferably, X and Y both contain aromatic groups. More preferably, X and Y are both tosyl groups, dansyl groups, tosyl or dansyl analogues, or substituted tosyl or dansyl groups.

A tosyl group has the structure:

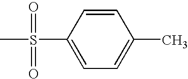

In general herein a tosyl analogue has the structure:

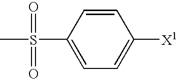

wherein X¹ represents optional substitution at the para ring position and more specifically has the structure:

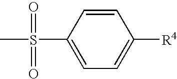

where R⁴ is an alkyl group having from two to 10 carbon atoms (preferably 2-6 carbon atoms).

A dansyl group has the structure:

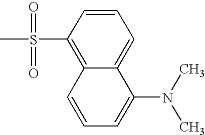

In general a dansyl analogue has the structure:

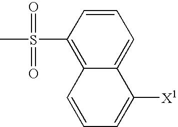

where X¹ represents optional substitution at the indicated ring positions and more specifically has the structure:

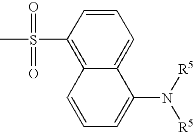

where R⁵ is an alkyl group having from two to 10 carbon atoms (preferably 2-6 carbon atoms).

In preferred compounds the Z groups are linked to the nitrogen of the macrocyclic ring via linker group L³ which can be an alkyl, carbonyl, or carboxy group (—L³—Z′). Preferred Z′ are optionally substituted alkyl groups, more preferably those having 5 or more carbon atoms, optionally substituted alkenyl groups, more preferably those having 5 or more carbon atoms, optionally substituted aryl groups, where Z′ is preferably not a fused group aryl group. More preferred L³ are alkyl and carboxy and more preferred Z′ are phenyl, cyclohexyl and cyclohexenyl.

All groups for W, X, Y and Z may be optionally substituted, for example, with polar substituents such as NH₂, N(R)₂, NO, NO₂, SH, SO₃H, SO₃R, OH, OR, COH, COR, CO₂H. and CO₂R (where each R independently is alkyl having one to 10 carbon atoms, and preferably one to 6 carbon atoms.) These polar groups are capable of aiding solubility of the compounds.

In a specific embodiment the methods of this invention employ one or more compounds of the formula:

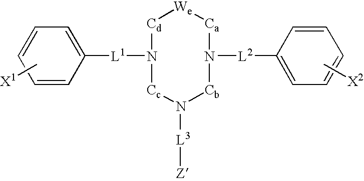

or pharmaceutically acceptable salts or solvates thereof,

where:

W represents a bridge carbon which is additionally bonded to one or two polar or non-polar side group substituents selected from the group consisting of double-bonded carbon which in turn is bonded to one or two hydrogens and/or R′ groups (i.e., ═CH₂, ═CRH, or ═C(R)₂), methylene halide (═CT₂); double bonded oxygen (═O); hydroxyl, optionally substituted alkyl of about one to 10 carbons (preferably of one to 6 carbons), optionally substituted alkenyl of about 2 to 10 carbon atoms (preferably of 2 to 6 carbon atoms); optionally substituted alkoxy of about one to 10 carbons (preferably one to 6 carbon atoms), optionally substituted aryl of about 6 to 10 carbons (preferably of about 7 to 10 carbons); halogen, methyl halogen(—CT₃, —CHT₂, or —CH₂T); epoxide (or oxirane); acyl (—CO—R); ester (—CO₂); CH₂OH and hydrogen; where halogen is F, Cl, I or Br; T, independently of other T, is F, Cl, I or Br, but preferably all T are the same halogen; R, independently of other R, is an optionally substituted alkyl, ether or thioether of about one to 10 carbons (preferably of one to 6 carbon atoms) or an aryl group of about 7 to 10 carbons (preferably of about 7 to 10 carbons) where R groups may be straight-chain, branched, cyclic or contain portions that are straight-chain, and/or branched, and/or cyclic, the R groups are optionally substituted and two R in the same group can form a cyclic moiety, L¹and L² independently can be —SO₂—, —SO—, —PO—, —PO(OH)—, —PO(H)—, —PO₂(OH)—, —PO₂(H)—, —PO₃(OH)—, carboxy (—OCO—), carbonyl (—CO—), or alkyl (e.g., —(CH₂)ₙ—where n is 1 or 2-; or one or both of L¹⁻² can be absent;

X¹ and X², independently, represent one or more non-hydrogen substituent groups on the aryl ring independent of ring position and generally can be any groups listed for optional substitution and more specifically can be one or more halogens, one or more alkyl groups, one or more alkoxy groups , one or more —SH or —SR groups, one or more ether groups; one or more thioether groups, one or more amino or alkyl-substituted amino groups, one or more nitro groups, one, two or more optionally substituted aryl groups one or both of which may be fused to the aryl ring shown in the formula; one or more hydroxyl groups, one or more acyl groups (—CO—R′), one or more ester groups (—CO₂R′), one or more —CO—NH—, or —CO—NR′, where R′, independently of other R′, is an alky of about one to 10 carbons or an aryl group of about 7 to 10 carbons (preferably of about 7 to 10 carbons and more preferably one to 3 carbon atoms). The alkyl group or alkoxy group substituent (X¹ or X²) on the aryl groups shown in the formula has from one to ten carbons (preferably from one to 6 carbon atoms) and is optionally substituted with one or more halogens or one or more hydroxyls;

L³ is a carbonyl (—CO—), carboxy (—OCO—) or an alkyl (e.g., —(CH₂)ₙ where n is 1 or 2;

Z′ represents a hydrogen, an optionally substituted alkyl group having from one to 10 carbon atoms, an optionally substituted alkene group having from two to ten carbons, an optionally substituted aryl group;

a and d, independently, represent a number from zero to 10; b and c, independently, represent a number from one to 10; and e represents a number from zero to three; and preferably, a+d+e ≧1, preferably e is 1, a and d are 1 to 3 and b and c are 2-4. The formula contains sufficient hydrogens for a stable molecule.

More specifically, triaza compounds of the following formula and pharmaceutically acceptable salts and solvates thereof can be employed in the methods of this invention.

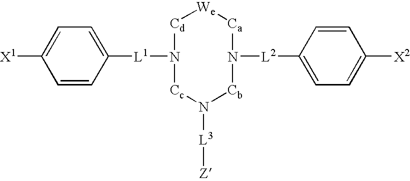

wherein W, a, b, c, d, e, L^{1-3,} and Z′ are as defined above in the previous formula and X¹ and X² represent para-substituents on the aryl rings illustrated in the formula and can be selected from polar and non-polar groups and may be, among others, alkyl, alkenyl, aryl, halogen, halogen-substituted alkyl, and amine substituents.

More specifically, triaza compounds of the following formula and pharmaceutically acceptable salts and solvates thereof can be employed in the methods of this invention.

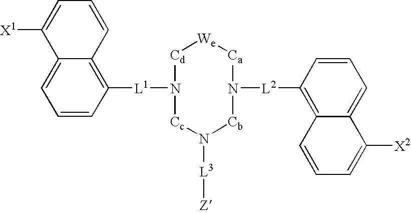

wherein W, a, b, c, d, e, L^{1-3,} and Z′ are as defined above in the previous formula and X¹ and X² represent para-substituents on the aryl rings illustrated in the formula and can be selected from groups listed below under optional substitution and preferably can be a halogen, a hydroxy, a hydroxyalkyl group, an alkyl group, a trihalomethyl group, an alkoxy group, or an amine or alkyl substituted amine group . Of particular benefit for use in the methods of this invention are compounds of the above formulas or pharmaceutically acceptable salts and solvates thereof wherein —L³—Z′ has the formula:

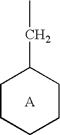

where the A ring is an optionally substituted phenyl ring, an optionally substituted cyclohexane ring or an optionally substituted cyclohexene ring.

In other specific embodiments triaza compounds of formulas:

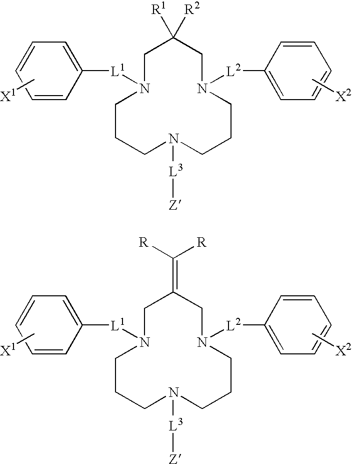

and salts and solvates thereof are useful in the methods of the present invention. In this formula, L¹⁻³, X¹⁻², and Z′ are as defined in previous formulas and R¹ and R² represent substituents on the central carbon of one of the carbon bridges. R¹ and R², independently, can be a hydrogen, hydroxyl, halogen, an optionally substituted alkyl group having one to 10 carbon atoms, an optionally substituted alkenyl group having 2 to 10 carbon atoms; optionally substituted alkoxy of about one to 10 carbons; methyl halogen(—CT₃, —CHT₂, or —CH₂T); epoxide (or oxirane); acyl (—CO—R); ester (—CO₂—R); CH₂OH and hydrogen; or R¹ and R² together can represent a double-bonded carbon which in turn is bonded to one or two hydrogens and/or R′ groups (i.e., ═CH₂, ═CRH, or ═C(R)₂), methylene halide (═CT₂);or a double bonded oxygen (═O). (preferably one to 6 carbon atoms), where halogen is F, Cl, I or Br; T, independently of other T, is F, Cl, I or Br, but preferably all T are the same halogen; R, independently of other R, is an optionally substituted alkyl, ether or thioether of about one to 10 carbons (preferably of one to 6 carbon atoms) or an aryl group of about 7 to 10 carbons (preferably of about 7 to 10 carbons) where R groups may be straight-chain, branched, cyclic or contain portions that are straight-chain, and/or branched, and/or cyclic, the R groups are optionally substituted and two R in the same group can form a cyclic moiety.

In yet more specific embodiments, compounds of the following formulas and pharmaceutically acceptable salts and solvates thereof are useful in the methods of this invention:

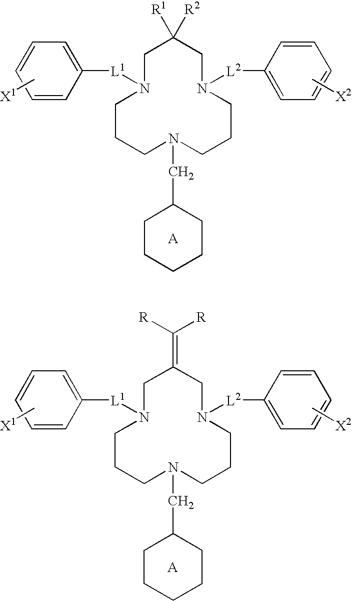

where X¹⁻², L¹⁻², R¹⁻², and R are as defined above and the A ring is an optionally substituted phenyl ring, an optionally substituted cyclohexane ring or an optionally substituted cyclohexene ring.

In additional specific embodiments, compounds of the following formulas and pharmaceutically acceptable salts and solvates thereof are useful in the methods of this invention:

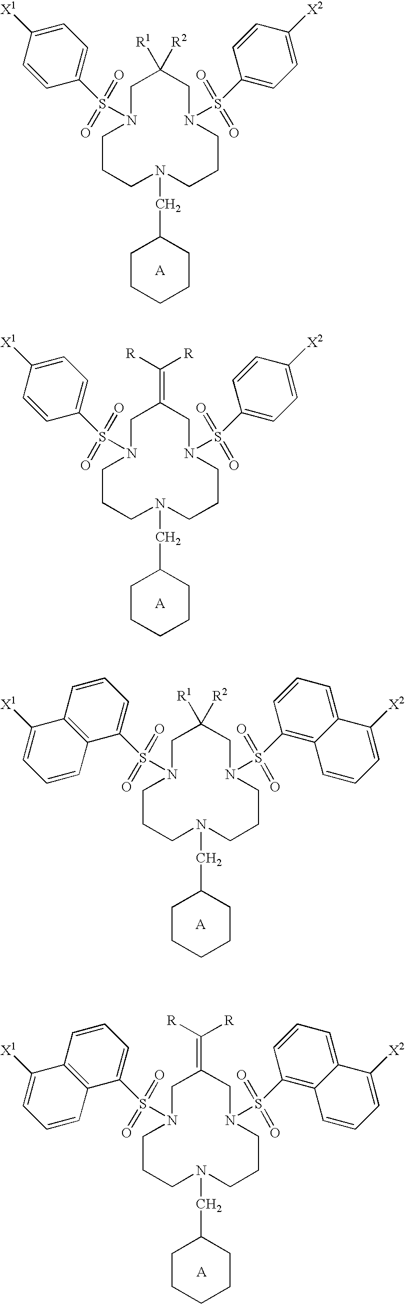

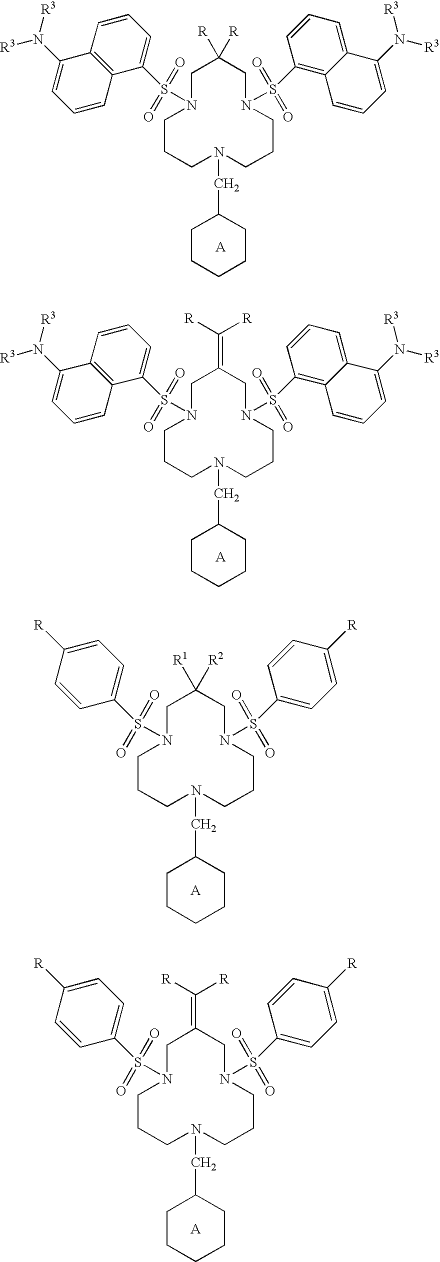

where X¹⁻², L¹⁻², R¹⁻², and R are as defined above and the A ring is an optionally substituted phenyl ring, an optionally substituted cyclohexane ring or an optionally substituted cyclohexene ring.

Representative Triaza Compounds Include:

3-Methylene-1,5-ditosyl-1,5,9-triazacyclododecane and pharmaceutically acceptable salts and solvates thereof;

5,9-Ditosyl-7-hydroxymethyl-1,5,9-triazabicyclo-[5,5,0]tridecane and pharmaceutically acceptable salts and solvates thereof,

5,9-Ditosyl-13-oxa-1,5,9-triazatricyclo[5,5,1^{1,7,}1^{7,12}]tetradecane and pharmaceutically acceptable salts and solvates thereof;

9-Benzyl-3-hydroxymethyl-1,5-ditosyl-1,5,9-triazacylododecane and pharmaceutically acceptable salts and solvates thereof;

9-Benzyl-3-chloromethyl-1,5-ditosyl-1,5,9-triazacyclododecane and pharmaceutically acceptable salts and solvates thereof;

3-Chloromethyl-1,5-ditosyl-1,5,9-triazacyclododecane and pharmaceutically acceptable salts and solvates thereof;

N,N-bis(3-toluenesulfonamidopropyl) toluenesulfonanide and pharmaceutically acceptable salts and solvates thereof;

1,5,9-Tritosyl-1,5,9-triazacyclododecane and pharmaceutically acceptable salts and solvates thereof;

3-Methylene-1,5,9-tritosyl-1,5,9-triazacyclododecane and pharmaceutically acceptable salts and solvates thereof;

3-Hydroxymethyl-1,5,9-tritosyl-1,5,9-triazacyclododecane and pharmaceutically acceptable salts and solvates thereof;

3-Chloromethyl-1,5,9-tritosyl-1,5,9-triazacylododecane and pharmaceutically acceptable salts and solvates thereof;

11-Methylene-1,5,9-triazabicyclo[7,3,3] pentadecane and pharmaceutically acceptable salts and solvates thereof;

1,5,9-Triazabicyclo[9,1,1]tridecane and pharmaceutically acceptable salts and solvates thereof;

9-Benzyl-3-keto-1,5-ditosyl-1,5,9-triazacyclododecane and pharmaceutically acceptable salts and solvates thereof;

9-Benzyl-3-methyl-1,5-ditosyl-1,5,9-triazacyclododecane and pharmaceutically acceptable salts and solvates thereof;

9-Benzyl-3-methylene-1,5-ditosyl-1,5,9-triazacyclododecane-9-oxide and pharmaceutically acceptable salts and solvates thereof;

9-Acyl-3-methylene-1,5-ditosyl-1,5,9-triazacyclododecane and pharmaceutically acceptable salts and solvates thereof;

9-Alkyl-3-methylene-1,5-ditosyl-1,5,9-triazacyclododecane and pharmaceutically acceptable salts and solvates thereof;

9-Acyl-3-methylene-1,5-ditosyl-1,5,9-triazacyclododecane epoxide and pharmaceutically acceptable salts and solvates thereof;

9-Benzyl-1-formyl-3-methylene-1,5,9-triazacyclododecane;

9-Benzyl-1-formyl-3-methylene-5-tosyl-1,5,9-triazacyclododecane and pharmaceutically acceptable salts and solvates thereof;

9-Benzyl-3-methylene-1-tosyl-1,5,9-triazacyclododecane and pharmaceutically acceptable salts and solvates thereof;

9-Benzyl-3-methylene-1-acyl-5-tosyl-1,5,9-triazacyclbdodecane and pharmaceutically acceptable salts and solvates thereof;

9-(Ethoxycarbonyl)-3-methylene-1,5-ditosyl-1,5,9-triazacyclododecane and pharmaceutically acceptable salts and solvates thereof;

N-Benzyl bis (3-benzenesulfonarnidopropyl) amine and pharmaceutically acceptable salts and solvates thereof;

N-Benzyl-3-methylene-1,5-dibenzenesulfonyl-1,5,9-triazacyclododecane and pharmaceutically acceptable salts and solvates thereof;

N-Benzylbis [3-(N′-2-propenyltoluenesulfonamido) propyl]amine dihydrogen Sulfate and pharmaceutically acceptable salts and solvates thereof;

N-Benzyl-N-[3-(N′-2-methyl-2-propenyl-toluenesulfonamido) propyl]—N—(3—toluenesulfonamido-propyl) amine dihydrogen sulfate and pharmaceutically acceptable salts and solvates thereof

N-Benzylbis[3-(N′-2-methyl-2-propenyltoenesulfonamido)propyl] amine dihydrogen sulfate and pharmaceutically acceptable salts and solvates thereof;

Specific compounds for use in the present methods include compounds 1,12-17, 19, 21-24 and 26, the structures of which are illustrated in Scheme 2, and pharmaceutically acceptable salts and solvates thereof.

1. 9-benzyl-3-methylene-1,5-ditosyl-1,5,9-triazacyclodecane (CADA) and pharmaceutically acceptable salts and solvates thereof;

12. 3-Methylene-1,5-ditosyl-1,5,9-triazacyclododecane and pharmaceutically acceptable salts and solvates thereof;

13. 5,9-Ditosyl-7-hydroxyinethyl-1,5,9-triazabicyclo-[5,5,O]tridecane and pharmaceutically acceptable salts and solvates thereof;

14. 5,9-Ditosyl-13-oxa-1,5,9-triazatricyclo[5,5,1^{1,7},1^{7,12}]-tetradecane and pharmaceutically acceptable salts and solvates thereof;

15. 9-Benzyl-3-hydroxymethyl-1,5-ditosyl-1,5,9-triazacyclododecane and pharmaceutically acceptable salts and solvates thereof;

16. 9-Benzyl-3-chloromethyl-1,5-ditosyl-1,5,9-triazacyclododecane and pharmaceutically acceptable salts and solvates thereof;

17. 3-Chloromethyl-1,5-ditosyl-1,5,9-triazacyclododecane and pharmaceutically acceptable salts and solvates thereof;

19. 1,5,9-Tritosyl-1,5,9-triazacyclododecane and pharmaceutically acceptable salts and solvates thereof;

21. 11-Methylene-1,5,9-triazabicyclo[7,3,3]pentadecane and pharmaceutically acceptable salts and solvates thereof;

22. 3-Methylene-1,5,9-tritosyl.-1,5,9-triazacyclododecane and pharmaceutically acceptable salts and solvates thereof;

23. 3-Hydroxymethyl-1,5,9-tritosyl-1,5,9-triazacyclododecane and pharmaceutically acceptable salts and solvates thereof;

24. 3-Chloromethyl-1,5,9-tritosyl-1,5,9-triazacyclododecane and pharmaceutically acceptable salts and solvates thereof; and

26. 1,5,9-Triazabicyclo [9,1,1] tridecane and pharmaceutically acceptable salts and solvates thereof.

Additionally representative triaza compounds of this invention are illustrated in Schemes 2 and 3.

Other preferred triaza compounds which can be used in the instant methods are symmetrical analogues of CADA, designed to have enhanced water solubility or to be capable of modification of biomolecules by electrostatic or hydrophobic interaction at the double bond position for reversible binding of proteins; for example:

41. 9-Benzyl-3-keto-1,5-ditosyl-1,5,9-triazacyclododecane and pharmaceutically acceptable salts and solvates thereof;

42. 9-Benzyl-3-methyl-1,5-ditosyl-1,5,9-triazacyclododecane and pharmaceutically acceptable salts and solvates thereof;

43. 9-Benzyl-3-methylene-1,5-ditosyl-1,5,9-triazacyclododecane-9-oxide and pharmaceutically acceptable salts and solvates thereof;

44. 9-Acyl-3-methylene-1,5-ditosyl-1,5,9-triazacyclododecane and pharmaceutically acceptable salts and solvates thereof;

45. 9-Alkyl-3-methylene-1,5-ditosyl-1,5,9-triazacyclododecane and pharmaceutically acceptable salts and solvates thereof; and

46. 9-Acyl-3-methylene-1,5-ditosyl-1,5,9-triazacyclododecane epoxide and pharmaceutically acceptable salts and solvates thereof.

Another series of compounds which can also be used in the instant methods is illustrated by formula II.

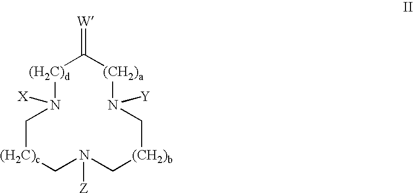

These compounds include:

50. 9-Benzyl-1-formyl-3-methylene-1,5,9-triazacyclododecane and pharmaceutically acceptable salts and solvates thereof;

51. 9-Benzyl-1-formyl-3-methylene-5-tosyl-1,5,9-triazacyclododecane and pharmaceutically acceptable salts and solvates thereof;

52. 9-Benzyl-3-methylene-1-tosyl-1,5,9-triazacyclododecane and pharmaceutically acceptable salts and solvates thereof; and

53. 9-Benzyl-3-methylene-1-acyl-5-tosyl-1,5,9-triazacyclododecane and pharmaceutically acceptable salts and solvates thereof.

Other compounds which can be employed in the instant methods include macrocyclic triamines of varying ring size, e.g., compounds 55 and 56 (Scheme 2).

Moreover, bicyclic analogues that can be represented by formulas 34-36 can also be used in the instant methods. Examples of bicyclic compounds of this invention are compounds 67-68 (Scheme 2).

Still other compounds which can be employed in the present methods include: 9-(Ethoxycarbonyl)-3-methylene-1,5-ditosyl-1,5,9-triazacyclododecane; 9-Benzyl-3-methylene-1,5-dibenzenesulfonyl-1,5,9-triazacyclododecane; and pharmaceutically acceptable salts and solvates thereof.

The synthesis of triaza compounds is described in U.S. Pat. Nos. 5,663,161, 6,342,492 and U.S. application Ser. No. 09/769,021, publication no. 2002/0019423 published Feb. 14, 2002, the entirety of each of which is incorporated herein by reference. These methods along with methods provided herein and methods that are well-known in the art can be employed by one of ordinary skill in the art to synthesize the triaza compounds of this invention.

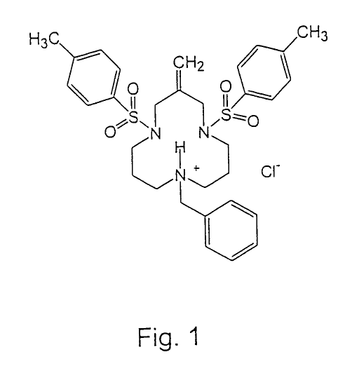

Particularly preferred compounds for use in the present methods include those listed in Table 1, including cyclotriazadisulfonamide (CADA) (depicted in FIG. 1), QJ023, QJ027, QJ028, QJ029, QJ030, QJ033, QJ035, QJ036, QJ037, QJ038, QJ040 and QJ041 whose structures are illustrated in Table 1.

A triaza compound described above can be processed in accordance with conventional methods of pharmacy to produce medicinal agents for administration to patients, e.g., mammals including humans and primates. The compound can be employed in admixture with one or more conventional pharmaceutically acceptable diluents, excipients, carriers and other appropriate components.

In accordance with the present invention, a triaza compound may be administered to a subject in need thereof via parenteral, oral, enteral or topical route. The administration route to be employed depends generally on the disorder, disease or condition to be treated as well as the type of individual to be treated and the tissue, or organ that is involved. Generally, parenteral is preferred. Generally, the triaza compounds are dispensed in unit dosage form comprising 10 to 1000 mg in a pharmaceutically acceptable carrier per unit dosage. The amount of a triaza compound administered to be therapeutically effective, i.e., sufficient to reduce the CD4 expression on helper T cells thereby ameliorating the symptoms of the pathological condition, depends on the nature of the pathological condition, the route of administration, as well as the weight and conditions of the subject. Therapeutic effectiveness of a given amount or form of pharmaceutical composition of this invention can also be assessed by the return to normal either partially or completely of one or more physiological or biochemical parameters associated with or causative of the disease or disorder. The precise amount can be determined by physicians or veterinary physicians. As a general rule, a triaza compound can be administered to a subject at about 0.1 to 100 mg/kg body weight/day, preferably 0.1 to 20 mg/kg/day to a human patient.

Pharmaceutical compositions of this invention comprise the active ingredient in a pharmaceutically acceptable carrier. The compositions may also include excipients and diluents. Suitable pharmaceutically acceptable carriers, excipients, and diluents include lactose, dextrose, sucrose, sorbitol, mannitol, starches, gum acacia, calcium phosphates, alginate, tragacanth, gelatin, calcium silicate, microcrystalline cellulose, polyvinylpyrrolidone, cellulose, water syrup, methyl cellulose, methyl and propylhydroxybenzoates, talc, magnesium stearate and mineral oil. The compositions can additionally include lubricating agents, wetting agents, emulsifying and suspending agents, preserving agents, sweetening agents or flavoring agents. The compositions of the invention may be formulated so as to provide controlled release (e.g., quick, sustained or delayed release) of the active ingredient after administration to the individual.

Triaza compounds can also be used in vitro or ex vivo to contact T cells isolated from any mammalian subject (including humans and primates) to generate a T-cell population with reduced CD4 expression.

TABLE 1

| | |
|---|---|
| 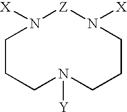 general fomula | 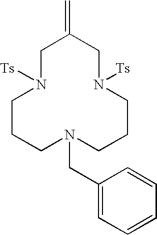 CADA |

| Compound | X | Y | Z |
|---|---|---|---|
| CADA | Ts | Bn | A = 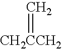 |
| QJ023 | Ts | 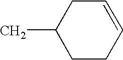 | A |
| QJ028 | Ts | 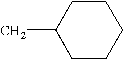 | A |
| QJ029 | Ts | CH₂CH₂CH₃ | A |
| QJ037 | Ts | 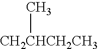 | A |
| QJ035 | Ts | CH(CH₃)₂ | A |
| QJ036 | Ts | 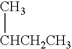 | A |
| QJ038 | Ts | CH₂CH₂CH(CH₃)₂ | A |
| QJ041 | Ts | 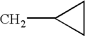 | A |
| QJ040 | Ts | 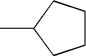 | A |
| QJ027 | Ts | 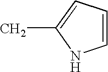 | A |
| QJ030 | Ts | 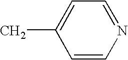 | A |
| QJ033 | Ts | 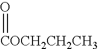 | A |

Ts (p-toluenesulfonyl) = 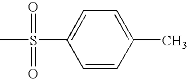
Bn (benzyl) = 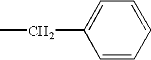 (CH₂Ph)

The invention is further illustrated by the following non-limiting examples.

### THE EXAMPLES

### Example 1

CADA Specifically Down-Modulates CD4 Expression

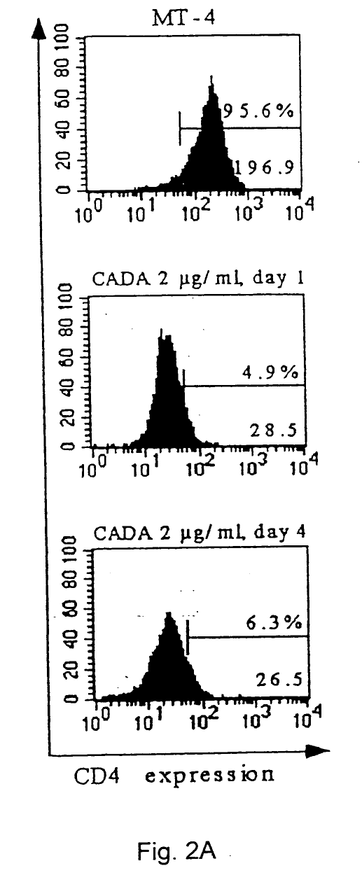

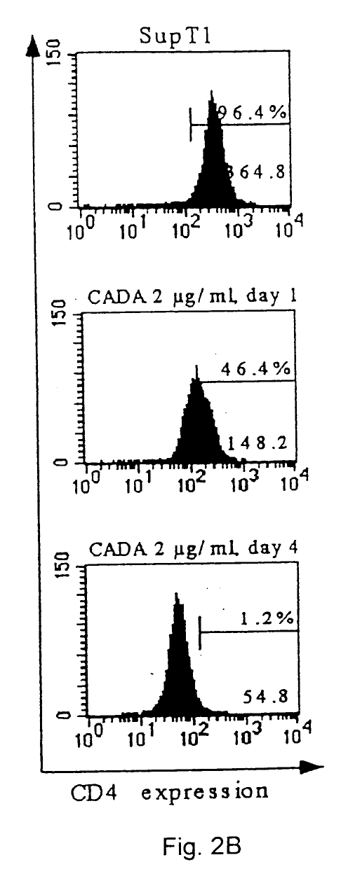

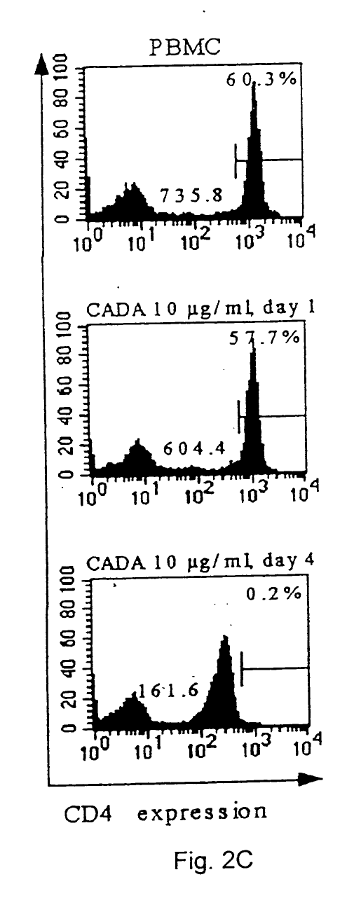

Cells were incubated with CADA hydrochloride and the CD4 expression on the cell surface was analyzed. As shown in FIG. 2, surface CD4 antigen expression decreased from 95.6% to 4.9% and 6.3% in MT-4 cells after incubation with CADA (3.2 μM) for 1 and 4 days, respectively. Among SupT1 cells, 96.4% expressed CD4, but this decreased to 46.4% and 1.2% when incubated with CADA (3.2 μM) for 1 and 4 days, respectively. In peripheral blood mononuclear cells (PBMCs), the percentage of CD4 expression decreased to 0.2% after incubation with CADA (16 μM) for 4 days (FIG. 2). Thus, CADA significantly down-modulated CD4 expression when evaluated in two different human T cell lines and in PBMCs. Comparable results were obtained with two other anti-CD4 monoclonal antibodies (mAbs), OKT4A and OKT4, which bind to different regions of CD4.

Expression of various other surface antigens was also examined in MT-4 cells, SupT1 cells and PBMCs after incubation with CADA (16 μM) or CADA-free medium at 37° C. for 4 days. After washing with phosphate-buffered saline (PBS) containing 2% FCS(fetal calf serum), the cells were incubated with mAb (as indicated in Table 1) for 20 min at 4° C. Then the cells were washed, fixed with 1% formaldehyde and analyzed by flow cytometry. All mAbs were purchased from Becton-Dickinson (Erembodegem, Belgium). As shown in Table 2, among all the surface antigens examined, only CD4 was markedly reduced after treatment of the cells with CADA for 4 days. Cell surface expression of the HIV coreceptors CXCR4 and CCR5 also remained unchanged after incubation of the cells with CADA for 4 days.

TABLE 2
Cell surface antigen expression in MT-4 T-cells,
SupT1 T-cells and PBMCs cultured for 4 days in
the presence or absence (control) of CADA.

| surface | MT-4 | | SupT1 | | PBMC | |
|---|---|---|---|---|---|---|
| molecule | control | CADA | control | CADA | control | CADA |
| CD2 | 1.5 | 2.6 | 2.4 | 0.5 | 96.2 | 90.7 |
| CD3 | 2.3 | 5.9 | 1.4 | 2.7 | 89.7 | 89.4 |
| CD4 | 95.6 | 6.3 | 96.4 | 1.2 | 60.3 | 0.2 |
| CD5 | 99.6 | 99.1 | 96.1 | 93.9 | 82.0 | 80.6 |
| CD8 | 2.8 | 3.5 | 98 | 92.3 | 25.3 | 25.1 |
| CD11b | 7.0 | 9.7 | 0.5 | 0.6 | 19.9 | 12.3 |
| CD25 | 96.7 | 93.3 | 2.3 | 1 | 37.6 | 35.5 |
| CD26 | 1.4 | 3.2 | 0.2 | 0.3 | 1.4 | 3.2 |
| CD28 | 6.6 | 6.6 | 98.7 | 90.8 | 92.0 | 89.6 |
| CD38 | 95 | 84.5 | 99.5 | 99.1 | 78.9 | 73.2 |
| CD45 | 2.65 | 3.57 | 99.5 | 99.6 | 98.9 | 98.3 |
| CD45RA | 2.7 | 2.0 | 2.4 | 0.5 | 69 | 64.3 |
| CD57 | 0.7 | 1.5 | 73 | 82.4 | 4.9 | 2.3 |
| CD71 | 85.2 | 98.8 | 39.8 | 33.9 | 29.3 | 27.6 |
| HLA-DR | 98.7 | 98.7 | 1.9 | 1.7 | 28 | 27 |
| TCRα/β | 0.8 | 1.1 | 2.5 | 1.6 | 80.4 | 82.3 |

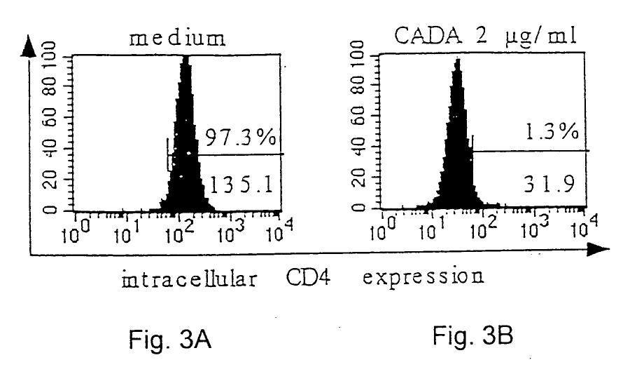

The effect of CADA on the intracellular presence of CD4 was also investigated. As shown in FIGS. 3A and B, the percentage of intracellular CD4 expression was 97.3% in untreated SupT1 cells (FIG. 3A), whereas it decreased to 1.3% when treated with CADA (3.2 μM) for 4 days (FIG. 3B). Similar results were also obtained in MT-4 cells and in PBMCs, and with two other anti-CD4 mAbs (OKT4A and OKT4).

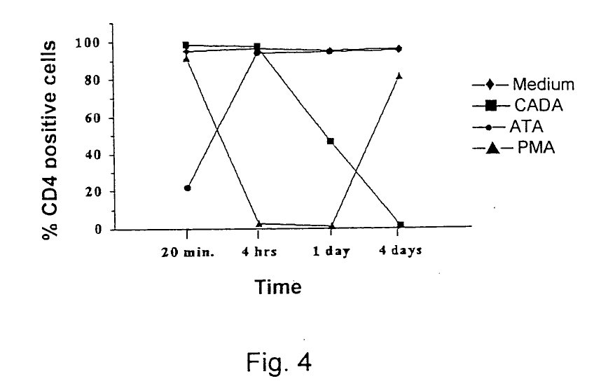

The anionic polymer aurintricarboxylic acid (ATA) is known to directly inhibit the binding of the OKT4AILeu3a mAb to CD4 (Schols, D., et al., Proc. Nat'l. Acad. Sci. U.S.A. 86:3322-3326 (1989)). Phorbol myristate acetate (PMA) is known to down-regulate CD4 expression by activating PKC at both the transcription and translation levels (Neudorf, S., et al., J. Clin. Lab. Anal. 3: 312-315 (1989); Chowdhury, I. H., et al., Virology 176: 126-132 (1990)). CADA was compared with ATA and PMA with regard to their down-modulating effects. As shown in FIG. 4, when SupT1 cells were incubated with ATA (24 μM) for 20 minutes, the percentage of cell surface CD4 expression decreased to 21.6%. The cell surface CD4 expression of ATA-treated SupT1 cells recovered quickly to normal levels after a 4-hour incubation period. After incubation with PMA (8 nM) for 20 minutes, there was only a slight CD4 decrease. However, CD4 expression was dramatically decreased after incubation with PMA for 4 hours. CD4 expression was almost undetectable in SupT1 cells when incubated with PMA for 1 day, but thereafter, the CD4 expression went up again to 81.6%. As compared to ATA and PMA, the down-modulation of CD4 by CADA had a different time-dependence effect. The percentage of CD4 expression in SupT1 cells treated with CADA (16 μM) after 20 minutes and after 4 hours were still as high as in the untreated cells. A CD4 decrease became clearly visible after incubation with CADA for 1 day. At day 4, the CD4 expression in SupT1 cells treated with CADA reduced to 1.2%. In addition, the PKC blocker staurosporine, which blocks PMA-induced effect on down-modulation of CD4, did not show any effect on the down-modulation of CD4 with CADA. These results indicate that the mechanism of CD4 down-modulation by CADA is different from that by PMA and ATA.

Gangliosides which are acidic glycolipids can also induce a selective loss of CD4 without affecting other surface molecules and can block CD4-mediated HIV-1 infection (Chieco-Bianchi, L., et al.(1989). CD4 modulation and inhibition of HIV-1 infectivity induced by monosialoganglioside GM1 in vitro. AIDS 3, 501-507.) Yet, the effects of gangliosides on CD4 are neutralized in the presence of serum albumin (Chieco-Bianchi, L., et al. 1989). The effect of human serum on CADA was studied, and it was found that the presence of human serum did not affect the CD4 down-modulation induced by CADA.

Compounds QJ023, QJ027, QJ028, QJ029, QJ030, QJ033, QJ035, QJ036, QJ037, QJ038, QJ040 and QJ041 (Table 1) were also shown to down-regulate CD4 expression.

### Example 2

The Antiviral Activity of CADA Against HIV-1 and HHV-7

The antiviral activity of CADA against HIV-1 and RHV-7 was investigated. PBMCs and CD4+T cell-depleted PBMCs (removed with CD8 magnetic beads; Dynal AS, Oslo, Norway), previously activated with purified PHA (2 μg/ml) and rhIL-2 (1 ng/ml) for 3 days, were pelleted and exposed to HIV-1 (1 ng p24 Ag per 106 cells) for 2 hours at 370C. Then the cells were washed three times with warm medium and cultured in complete medium containing rhIL-2 (1 ng/ml) in the presence or absence of different concentrations of CADA hydrochloride. The anti-HIV-1 assays were also performed in MT-4 and SupT1 cells. CADA hydrochloride was diluted in the plates, then the MT-4 cells or SupT1 cells were added and finally the T-tropic HIV-1 strains. The supernatant was collected at day 5 for MT-4 cells, day 8 for SupT1 cells and day 12 for PBMCs, and stored at −20° C. and analyzed for HIV-1 core antigen by p24 Ag ELISA (NEN, Brussels, Belgium). The SupT1 cells were refreshed on day 4, and half of the medium and cells were replaced and fresh CADA-free medium was added. For the PBMCs, fresh culture medium was added at day 6 containing rhIL-2 (but no CADA). For the anti-HHV-7 assays in PBMCs, CADA hydrochloride was diluted in a plate, 2×10˜cells were added and then HHV-7 (KHR strain) was added in complete medium containing rhIL-2 (1 ng/ml). Fresh culture medium was added again at day 6 containing rhIL-2 (but no CADA). The same infection protocol was used for the SupT1 cells, but without rhIL-2. The SupT1 cells were refreshed on day 4 and day 8, half of the medium and the cells were replaced, and fresh medium without CADA was added. The HHV-7 antigen expression was monitored (at day 12) with a specific mAb to HHV-7 (RK-4) (Advanced Biotechnologies, Columbia, Md.) by flow cytometry, as described previously (Thang, Y., et al., Antiviral Res. 43:23-35 (1999)).

As shown in Table 3, the inhibitory concentration (IC₅₀) of CADA for HHV-7 ranged between 0.3-1.5 μM when evaluated in SupT1 cells, PBMCs and CD8+ T-cell depleted PBMCs. The IC₅₀ values of CADA against several CXCR4-using (X4) HIV-1 strains (IIIB, NL4.3, RF) were between 0.3-3.1 μM. The IC₅₀ of CADA against the CCR5-using (R5) HIV-1 strain BaL was 0.8 μM in PBMCs. The cytotoxic concentration (CC50) values of CADA in MT-4 cells, SupT1 cells and PBMC were 134 μM, 185 μM and 73 μM, respectively. The IC₅₀ of PMA for the HIV-1 strains (IIIB, RF and NL4.3) in MT-4 cells was about 0.3 nM, and the IC₅₀ for HHV-7 infection in SupT1 cells was 0.6 nM. The CC₅₀ value of PMA in MT-4 and SupT1 cells was 2.1 nM and 1.5 nM, respectively. Thus, the selectivity index (SI), or ratio of CC₅₀ to IC₅₀, was very low for PMA (average, 2-7), while CADA had an SI varying from 40 to 620.

TABLE 3
Antiviral activity of CADA hydrochloride against HHV-7 and HIV-1

| | IC₅₀(μM) | | | |
|---|---|---|---|---|
| Virus strain | MT-4 | SupT1 | PBMC | CD8⁺ depleted PBMC |
| HHV-7 | NA* | 0.3 | 1.5 | 0.9 |
| HIV-1 strains | | | | |
| III_{B} | 0.3 | 0.9 | 0.6 | ND^{†} |
| RF | 0.9 | 1.8 | 1.9 | ND^{†} |
| NL4.3 | 0.5 | 3.1 | 1.6 | ND^{†} |
| BaL | NA* | NA* | 0.8 | ND^{†} |

*Not applicable.
^{†}Not determined.

Table 4 presents the results for MT-4 cells pretreated with CADA for 1 day at 8.1 μM and then infected with the HIV-1 strain NL4.3 or RF in the presence or absence of CADA. HIV-1 infection in CADA-pretreated MT-4 cells was decreased, as compared to the untreated MT-4 cells (p24 core antigen: 158627 pg/ml versus 34839 pg/ml). In addition, when the cells were pretreated with CADA, administration of CADA at 0.6 μM then completely blocked the viral replication. Comparable results were also obtained with SupT1 cells and PBMCs. Thus, as expected from the data presented in FIG. 2, pre-treatment of the cells with CADA clearly enhanced its antiviral potency. As three CD4 binding events are needed to efficiently activate HIV-1 Env trimers (Layne, S. P., et al., Nature 346: 602-605 (1990)), multimeric CD4 binding is required for HIV infection, further confirming that receptor density plays a crucial role in the efficiency of viral infectivity (Platt, E. J., et al., J. Virol. 71: 883-890 (1997)). Data obtained on the quantification of the CD4 receptor assessed by antibody binding showed that after treatment with CADA the amount of CD4 Ab bound to the cells dropped to approximately 5,000/cell which represent an almost 90% decrease in bound antibody. Primary HIV isolates seem to be much more dependent on the level of CD4 expression (Kabat; D., et al., J. Virol. 68:2570-2577 (1994)). When evaluated against 6 different primary HIV-1 isolates in PBMCs, CADA showed a potent (but variable) activity ranging from 0.002-2.3 μM. Although CD4-independent viruses are described (Hofflian, T. L., et al., Proc. Natl. Acad. Sci. U.S.A. 96: 63 59-6364 (1999)), these are much more sensitive to neutralization by antibodies, which explains the rarity of CD4-independent wild-type HIV variants (Kolchinski, P., et al., J. Virol. 75: 204 1-2050 (2001)).

TABLE 4
Levels of p24 viral core antigen (pg/ml) in the supernatant
of MT-4 cells infected with HIV-1 strains NL4.3 or RF.

| | | NL4.3 | | RF | |
|---|---|---|---|---|---|
| | | pre-treated* | untreated | pre-treated* | untreated |
| Medium | | 34839 | 158627 | 42853 | 100002 |
| CADA | (3.2 μM) | <5 | <5 | <5 | <5 |
| | (0.6 μM) | <5 | 63098 | <5 | 56139 |
| | (0.13 μM) | 33785 | 127838 | 35894 | 75118 |

*Cells were pre-treated with CADA hydrochloride (8.1 μM) for 24 hours, then more CADA was added again after HIV infection at the indicated concentrations. The supernatant was collected and analyzed for HIV-1 core antigen by p24 Ag ELISA.

### Example 3

CD4 down-modulating activity of CADA analogs.

Compounds and monoclonal antibodies (mAbs). CADA and CADA derivatives were synthesized by as described in examples herein. The structures of the different CADA analogies are shown in Scheme 2. Each compound was dissolved at 10 mg/ml in DMSO.

The monoclonal antibody (mAb) CD4 (SK3) was purchased from Becton Dickinson Biosciences (Erembodegem, Belgium). The HIV-1 p24 antigen ELISA kit was purchased from NEN (Brussels, Belgium). The specific mAb to HHV-7 (RK-4) (Advanced Biotechnologies, Columbia, Md.) recognizing an early HHV-7 protein was used to detect HHV-7-infected cells.

Viruses and Cell Cultures. The HIV-1 T-tropic (X4) molecular clone NL4.3 was obtained from the National Institute of Allergy and Infectious Disease AIDS reagent program (Bethesda, Md.). The KHR strain of HHV-7 was kindly provided by Dr. K Amanita (Department of Microbiology, Osaka University School of Medicine, Osaka, Japan). The CD4+T-cell lines MT-4 and SupT1 were obtained from the American Type Culture Collection (Rockville, MD) and cultured in RPMI 1640 medium (Gibco BRL, Gaithersburg, Md.) with 10% heat-inactivated fetal calf serum (FCS) (Biowhittaker, Belgium) and 2 mM L-glutamine (Gibco BRL, Gaithersburg, Md.). The cell cultures were maintained at 37° C. in a humidified, CO₂-controlled atmosphere and subcultivations were done every 2 to 3 days. The HHV-7 stock was made in the SupT1 cell line, whereas the HIV-1 stock was made in MT-4 cells.

Antiviral Assays. For HIV-1, MT-4 cells were infected with the HIV-1 strain NL4.3. Briefly, five-fold dilutions of the compounds (in 100 μl) were added to 96-well flat bottom plates (Iwaki, Japan). Then, to each well, 7.5×10⁴ MT-4 cells were added in 50 μl medium, followed by 50 μl of diluted HIV-1 stock (strain NL4.3). Cytopathic effect (CPE) induced by the virus was checked regularly microscopically. When strong CPE was observed (after 3 to 5 days of incubation) in untreated HIV-1 infected cells, the supernatant was collected, stored at −20° C. and analyzed for HIV-1 core antigen by p24 Ag ELISA (NEN™, PerkinElmer Life Sciences, Boston, Mass.). Finally, the IC₅₀ value of the compounds (i.e., the concentration of the compound required for 50% reduction of HIV infection as measured by viral p24 antigen level in the supernatant of infected MT-4 cells) was calculated.

For HHV-7, five-fold dilutions of the compounds were added in 500 μl culture medium in 24-well flat bottom plates (Iwaki, Japan), whereupon 2×10⁵ SupT1 cells were added in 400 μl culture medium. After 30 min incubation at room temperature, 100 μl of HHV-7 stock was added to each well. HHV-7-infected and mock-infected SupT1 cells were cultured in a final volume of 1 ml medium in the absence of the compounds. On day 4, half of the medium and the cells were replaced and fresh medium without new compound was added. This procedure was repeated every 2 or 3 days. The CPE was checked regularly microscopically. When CPE was observed, the HHV-7 antigen expression was monitored by flow cytometry, as described previously (Zhang, Y. et al. (1999) “Selective activity of various antiviral compounds against HHV-7 infection Antiviral Res. 43, 23-35.)

TABLE 5
Anti-HIV-1 and HHV-7 activity, and CD4 down-modulating capability
of the different CADA analogs in MT-4 and SupT1 cells.

| | MT-4 | | | SupT1 | | |
|---|---|---|---|---|---|---|
| | IC₅₀ (μg/ml) | | CC₅₀^{a} | IC₅₀ (μg/ml) | | CC₅₀ |
| Compound | CD4^{b} | HIV-1^{c} | (μg/ml) | CD4 | HHV-7^{d} | (μg/ml) |
| CADA | 0.35 | 0.72 | 23.6 | 0.57 | 0.35 | >50 |
| QJ023 | 0.26 | 0.29 | 22.2 | 0.36 | 0.10 | 28.9 |
| QJ027 | 3.87 | 4.40 | 20.2 | 6.02 | 3.10 | 21.7 |
| QJ028 | 0.21 | 0.20 | 20.7 | 0.25 | 0.05 | 28.1 |
| QJ029 | 2.58 | 2.00 | 20.7 | 3.06 | 0.91 | 22.2 |
| QJ030 | 2.57 | 2.36 | 21.3 | 7.24 | 1.59 | >50 |
| QJ033 | 1.19 | 1.22 | 25.5 | 1.89 | 0.73 | 32.5 |
| QJ035 | 6.06 | 5.55 | 22.2 | >10 | 1.40 | 23.0 |
| QJ036 | 1.16 | 0.95 | 20.9 | 1.79 | 1.19 | >50 |
| QJ037 | 0.53 | 0.48 | 22.1 | 0.69 | 0.13 | 24.0 |
| QJ038 | 0.41 | 0.31 | 19.4 | 0.48 | 0.34 | 8.4 |
| QJ040 | 5.34 | 4.23 | 17.0 | 7.91 | 1.55 | 20.5 |
| QJ041 | 3.48 | 3.66 | 18.6 | 4.15 | 1.76 | 21.9 |
| AS-N6P6 | 3.22 | 2.38 | 21.4 | 4.27 | 1.50 | >50 |
| 95-213 | 6.46 | 8.05 | >50 | 9.63 | 0.68 | >50 |
| 98-035 | 4.81 | 4.82 | >50 | 5.33 | 0.70 | >50 |
| HJC241 | 1.90 | >10 | 32.0 | >50 | 1.09 | >50 |
| HJC321 | 1.99 | 3.88 | 26.1 | 3.17 | 1.94 | >50 |
| AS117 | 1.12 | 1.15 | 30.7 | 1.49 | 0.78 | >50 |
| AS-PB127 | 1.41 | 1.40 | 35.7 | 1.77 | 0.88 | >50 |
| MFS-SC001 | 7.08 | >10 | 23.1 | 4.09 | 0.65 | 32.2 |
| 98-037 | >50 | >50 | >50 | >50 | >50 | >50 |
| AS112 | >10 | >10 | 27.8 | >50 | >50 | >50 |
| 97-269 | >10 | >10 | 27.9 | >50 | >50 | >50 |
| MFS010 | >10 | >10 | 29.3 | >50 | 4.43 | >50 |
| MFS117 | >10 | 10 | 18.9 | >50 | 0.92 | >50 |
| MFS105 | >50 | >50 | >50 | >50 | >50 | >50 |

^{a}CC₅₀: 50% cytotoxic concentration, or concentration of the compound required to reduce the viability of the cells by 50%.
^{b}IC₅₀ for CD4 down-modulation: concentration of the compound required for 50% inhibition of extracellular CD4 expression, as measured by flow cytometry.
^{c}IC₅₀ for HIV-1 (NL4.3) infection: concentration of the compound required to reduce viral HIV-1 recplication by 50% as measured by the p24 Ag ELISA.
^{d}IC₅₀ for HHV-7 infection: concentration of the compound required for 50% reduction of the HHV-7 antigen expression on SupT1 cells, as measured by flow cytometry.

Flow Cytometric Analyses. To study the effect of the CADA derivatives on surface CD4 antigen expression, MT-4 and SupT1 cells were incubated with a serial 5-fold dilution of the compounds (50, 10, 2, 0.4 and 0.08 μg/ml) or medium at 37° C. Cell surface CD4 antigen expression was analyzed at day 3 (MT-4) or day 4 (SupT1). Briefly, after washing with phosphate-buffered saline (PBS) containing 2% fetal calf serum (FCS), cells were incubated with FITC-conjugated anti-CD4 (SK3) mAb for 30 min at 4° C. As a negative control for a specific background staining, cells were stained in parallel with Simultest Control γ₁/γ^{2a} (Becton Dickinson). Then the cells were washed, fixed with 1% formaldehyde and analyzed by flow cytometry in a FACScalibur (Becton Dickinson, San Jose, Calif., USA). Data were acquired and analyzed with CellQuest software (Becton Dickinson). For the calculation of the CD4 receptor expression, the mean fluorescence intensity (MFI) of each sample was expressed as percentage of the MFI of control cells (after subtracting the MFI of the isotype control). Finally, the IC₅₀ value of the compounds (i.e., the concentration of the compound required for 50% inhibition of extracellular CD4 expression) was calculated.

Cytotoxicity Assay. Cellular toxicity of the compounds was measured by trypan blue exclusion and also by Propidium Iodide by flow cytometry after 3 or 4 days of incubation, in parallel with the CD4 antigen expression. The CC₅₀ value of each CADA analog is the concentration (in μg/ml) required to reduce the viability of the cells by 50%.

CD4 down-modulating activity of CADA analogs. The CD4 down-modulating activity of CADA and 30 other CADA derivatives are shown in Table 5. The prototype compound CADA markedly down-regulates CD4 receptor expression in MT-4 and SupT1 cells with a IC₅₀ of 0.35 and 0.57 μg/ml, respectively. Removal of two double bounds in the aromatic ring of the benzyl group of CADA, as in compound QJ023, slightly enhanced the CD4 down-modulating activity. If the benzyl group of CADA was replaced by a cyclohexylmethylene group, as done in compound QJ028, the CD4 down-regulating potency was somewhat more pronounced (2-fold more active than CADA). In fact, QJ028 was the most potent compound of the CADA derivatives tested so far. The cytotoxic concentration (CC₅₀) of QJ028 was 28.1 μg/ml in SupT1 cells, which is about a 100-fold higher than its IC₅₀ value (Table 5), resulting in a selectivity index (CC₅₀/IC₅₀) of approximately 100.

Inclusion of a nitrogen atom at position 3 or 4 of the benzene ring of the benzyl group (3-pyridinylmethylene and 4-pyridinylmethylene group in compound ASN6P6 and QJ030, respectively) had a detrimental effect on the CD4 down-modulating activity (respectively, 7- and 9-fold less active in MT-4 cells as compared to CADA). Furthermore, the presence of a nitrogen atom at position 2 of the aromatic group (2-pyridinylmethylene in compound 98-037) resulted in a complete loss of activity. In contrast, compound QJ027 which has a nitrogen atom at the same position (position 2) but in a smaller aromatic ring (2-pyrrolylmethylene group) still had a CD4 down-modulating potency, although 10-fold less as compared to CADA. However, smaller cyclic structures in substitution for the cyclohexylmethylene group of QJ028 reduced the activity of the compound (e.g. cyclopropylmethylene in compound QJ041).

The CD4 down-modulating potency of CADA was affected in a different way when the benzyl group was replaced by an aliphatic chain. Substitution by a longer open chain, such as an isopentyl group in compound QJ038, resulted in a similar CD4 down-regulating activity, however, compounds with a short chain (isopropyl in compound QJ035) were clearly less active than CADA (17-fold less active for the CD4 down-regulation in MT-4 cells and not active in SupT1 cells ). Analogs with an aliphatic chain made of 3 or 4 carbon atoms appeared to have a CD4 down-modulating activity somewhat in between (e.g. compounds QJ029 and QJ036). Interestingly, comparison of compound QJ036 with 98-035 let us suggest that the isobutyl group of compound 98-035, with its rigid tail of 2 methyl groups, probably has more difficulties to interact with its target molecule (98-035 is 4-fold less active than QJ036). Thus, the length of the aliphatic chain of the CADA derivatives seemed to be crucial for their CD4 down-regulating potency. This is further stressed by the comparison of the compounds QJ033, 95-213 and 97-269. Again, analog QJ033 with its longer propyloxycarbonyl group has a 5-fold higher CD4 receptor down-modulating activity than compound 95-213 with its shorter ethyloxycarbonyl group. Substitution by the short 9-acetyl group, as in compound 97-269, resulted in a complete loss of anti-CD4 activity.

Modifications of the 3-methylene group, that makes a rigid bridge between the two toluenesulfonyl groups of CADA, had various effects on the CD4 down-regulating potency. Removal of the methylene group (compound MFS-SC001) had a harmful effect on the down-regulating activity (20-fold less active than CADA in MT-4 cells). Furthermore, substitution of 3-methylene by 3-oxo (MFS010) resulted in significantly decreased CD4 down-regulating activity. If the rigid 3-methylene bridge was replaced by a “movable” 3-hydroxymethylene or a 3-chloromethylene linker (compound HJC321 and AS117, respectively), a slight diminution of the CD4 down-regulating potency was observed (6- and 3-fold, respectively).

Finally, the importance of the toluenesulfonyl groups was investigated. Removal of the benzene ring out of the tosyl structure (methanesulfonyl group in compound MFS105) or substitution of toluenesulfonyl by a larger group (butyloxymethylenephenylsulfonyl in compound MFS 117) resulted in significant loss of the CD4 down-regulating activity. The presence of a third tosyl structure instead of the 9-benzyl group (compound AS 112) had also a negative effect on the activity. Replacement of the methane group by a bromine atom as in compound AS-PB127 did not enhance the CD4 down-regulating activity (4-fold less active than CADA).

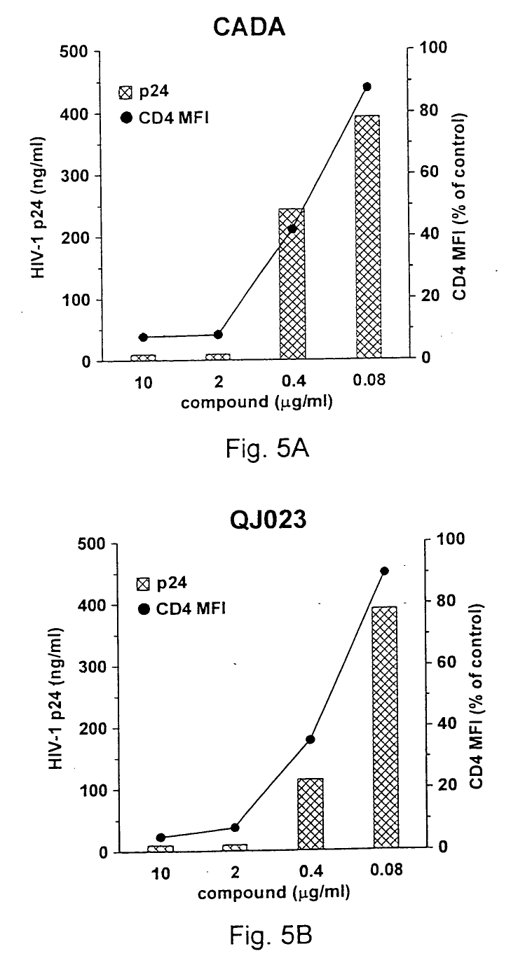

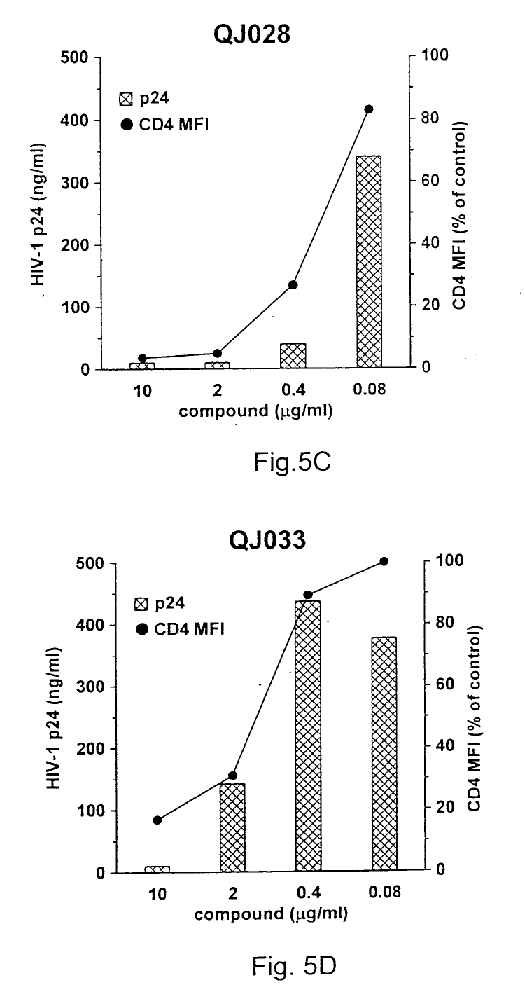

Antiviral activity of CADA analogs against HIV-1 and HHV-7. Table 5 also shows the anti-HIV-1 and anti-HHV-7 activity of the different CADA derivatives. The prototype compound CADA inhibits HIV-1 NL4.3 replication in MT-4 cells (IC₅₀═0.72 μg/ml), as well as HHV-7 infection in SupT1 cells (IC₅₀ ═0.35 μg/ml). As was the case for the CD4 down-regulating activity, compound QJ028 appeared to be the most active analog when tested for its antiviral potency. Also, the anti-HIV-1 and anti-HHV-7 data of derivative QJ023 correlate with the CD4 down-regulating activity of this compound. A dose response effect of CADA, QJ023, QJ028 and QJ033 on HIV infection and on CD4 down-modulation is combined in FIG. 5. MT-4 cells were treated with different doses of each compound (10, 2, 0.4 and 0.08 μg/ml). After 4 days of incubation, CD4 receptor expression was measured flow cytometrically. As shown in FIG. 5, CADA at a concentration of 10 and 2 μg/ml significantly down-modulated CD4 receptor expression, whereas at 0.4 μg/ml 58% down-regulation was detected. A lower dose of the compound (i.e. 0.08 μg/ml) had no inhibitory effect on CD4 receptor expression. When the anti-HIV-1 activity of CADA was measured in MT-4 cells, a similar dose-dependent effect of CADA on the NL4.3 infection was observed. Thus, MT-4 cells were infected with the HIV-1 strain NL4.3 in the presence of decreasing concentrations of each compound (10, 2, 0.4 and 0.08 μg/ml). After 4 days of incubation, when CPE was clearly visible, supernatant was collected and viral replication was measured by p24 Ag ELISA. High concentration of CADA (i.e. 10 and 2 μg/ml) resulted in a significant inhibition of viral replication (FIG. 5). CADA at a dose of 0.4 μg/ml resulted in a 25% inhibition of virus production, whereas a lower dose of the compound (i.e. 0.08 μg/ml) had no anti-HIV-1 activity as evident from the p24 core antigen values (p24 level was 392 ng/ml and that of infected control cells was 325 ng/ml). These results demonstrate that the CD4 down-regulating activity of CADA directly correlates with its anti-HIV potency. A similar correlation could be observed with other compounds (FIG. 5). Administration of 0.4 μg/ml QJ028 to the cell cultures resulted in 88% inhibition of NL4.3 infection and 73% down-regulation of the CD4 receptor. When compound QJ033 was tested for its antiviral activity and CD4 down-modulating potency, an inhibition of 56% and 69%, respectively, could be measured at a concentration of 2 μg/ml, whereas at 0.4 μg/ml no antiviral activity nor significant CD4 down-regulation was observed (FIG. 5). Also for the other CADA analogs, comparable IC₅₀ values were obtained for CD4 down-regulation and inhibition of virus replication (Table 5).

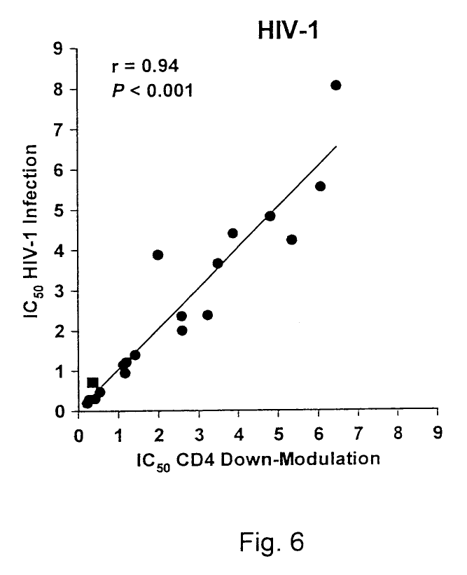

Correlation between HIV-1 or HHV-7 and CD4 down-modulation. The IC₅₀ values of the CADA derivatives for CD4 receptor down-modulation were compared with their IC₅₀ values for inhibition of HIV-1 replication. There was a close correlation among the inhibitory effects of the compounds on HIV-1 NL4.3 infection and CD4 receptor expression. When IC₅₀ values of the CADA analogs for HIV-1 replication were plotted as a function of their IC₅₀ values for CD4 down-regulation (on a linear-linear scale) (FIG. 6), linear regression showed a strong linear correlation (r=0.94) between the inhibitory effects on HIV-1 infection and on CD4 expression.

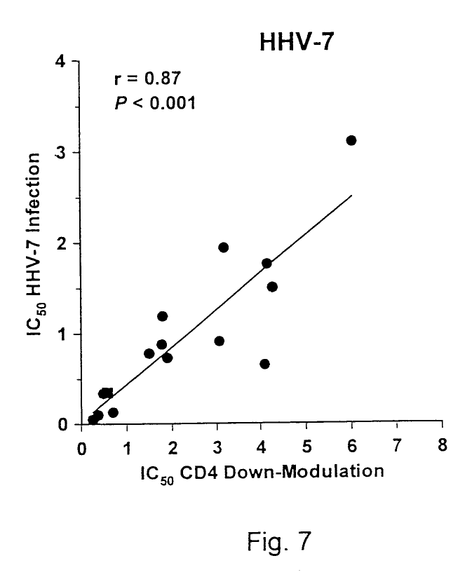

When the IC₅₀ values of the CADA analogs for HHV-7 replication were plotted as a function of their IC₅₀ values for CD4 receptor down-modulation in SupT1 cells (FIG. 7), linear regression again showed a close linear correlation (r=0.87) between the inhibitory effects on virus replication and on CD4 receptor expression.

### Example 4

Synthesis of Unsymmetric CADA Analogs

Unsymmetric triaza macrocycles of this invention are synthesized as illiustrated in the following scheme:

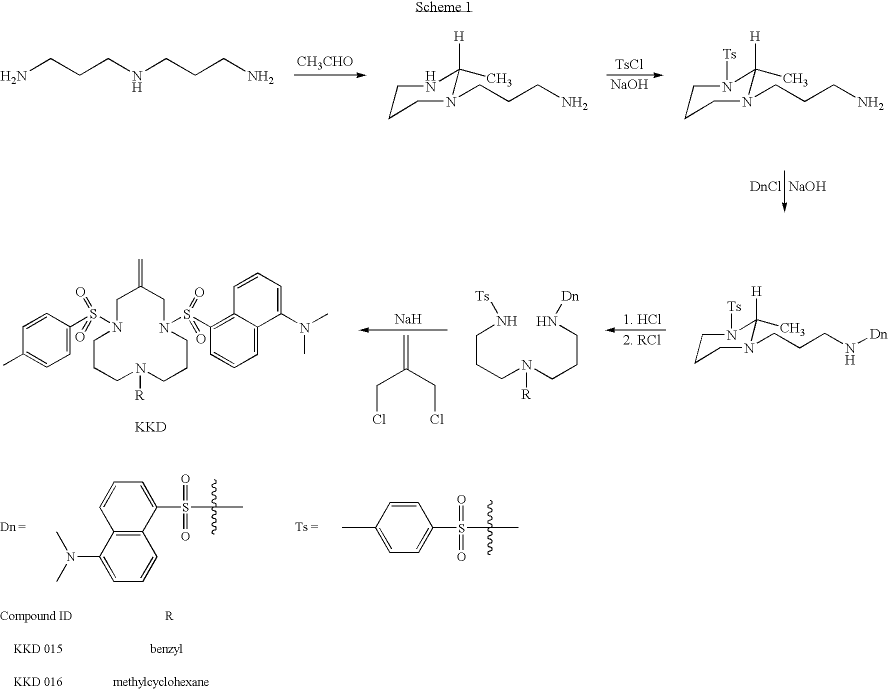

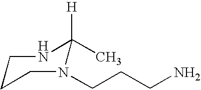

1-(3′-Aminopropyl)-2-methylhexahydropyrimidine

Into a two-necked 1.0 L round-bottomed flask equipped with a thermometer and an addition funnel was poured 22.1 g (0.168 mol) of N (3-Aminopropyl) 1,3-propanediamine and 800 mL of CHCl₃. The mixture was cooled to 3° C. in an ice-bath followed by the dropwise addition of 7.42 g (0.168 mol) of ethanol amidst stirring. After 5 more minutes of stirring, the solvent was removed by rotary evaporation followed by drying under high vacuum (0.5 mm). A crude solidifying liquid 25.54 g (97%) was obtained which was further purified by vacuum distillation at 110 mm and 100° C. to give 21.98 g (83%) of colorless oil as product.

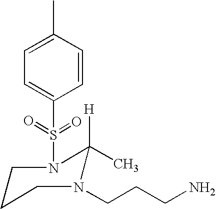

1-(3′-Amino propyl)-2-methyl-3-toluenesulfonamido hexahydropyrimidine

Into a 50 mL round-bottomed flask equipped with addition funnel was poured 1.14 g (7.3 mmol) of 1-(3′-Aminopropyl)-2-methylhexahydropyrimidine and 25 mL of 1:1:2 2M NaOH/THF/ water mixture. The addition funnel was charged with 1.4 g (7.3 mmol) of p-toluenesulfonyl chloride dissolved in 20 mL of 1:1:2 2M NaOH/THF/water mixture. The flask and its contents were cooled to 0° C. and the contents of the addition funnel added all at once. The reaction mixture was stirred overnight with the temperature rising to 23° C., after which it was saturated with NaCl. After decantation, the organic layer (top) was separated and dried with anhydrous Na₂SO₄. The solvent was removed by rotary evaporation followed by drying under high vacuum (0.5 mm) to give 0.8 g (35%) of solidifying oil.

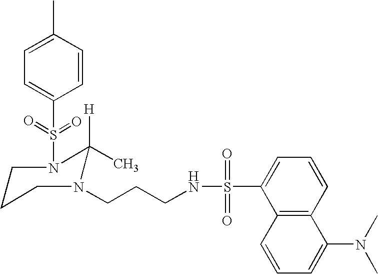

1(3′-5-Dimethylamino-1-naphthalenesulfonamidopropyl)-2-methyl-3-p-toluenesulfonamido hexahydropyrimidine

In a 500 mL round-bottomed flask, was dissolved 5.96 g (19.2 mmol) of 1-(3′-Amino propyl)-2-methyl-3-toluenesulfonamido hexahydropyrimidine and 5.17 g (19.2 mmol) of dansyl chloride in 120 mL of CH₂Cl₂. To the flask and its contents were added a further 120 mL of saturated aqueous solution of Na₂CO₃ and the reaction mixture stirred for 18 h. Sodium hydroxide (4.0 g, 0.1 mol) was added to the reaction mixture and stirring continued for 2 more hours. The organic (bottom) layer was separated and evaporated to dryness to give a mixture of compounds. Purification of the compound was done by column chromatography. A first silica gel column using 100% ethyl acetate, then 1:9 methanol/CHCl₃ solvent system removed dansyl and tosyl chloride impurities and eluted the ring-opened form of the desired product, which was still impure. A second column, using the same conditions as the first, purified the mixture further, but complete purification was not achieved. A crude yield of 5.0 g (50%) was achieved. Refluxing of the reaction mixture in M HCl and THF has been found to improve the yields of other analogs.

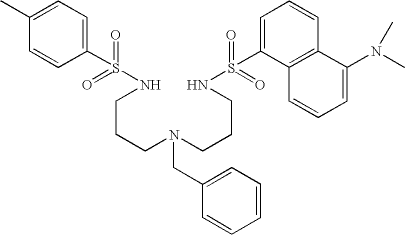

N-(3-(5-dimethylamino-1-napthalenesulfonamidopropyl)-N-(3-p-toluenesulfonamidopropyl) benzamine (KKD-014)

A mixture of 2.0 g (3.9 mmol) of N-(3-(5-dimethylamino-1-napthalenesulfonamidopropyl)-N -(3-p-toluenesulfonamidopropyl) amine, 35.0 mL of acetonitrile, 0.1 g (0.66 mmol) of sodium iodide, 0.40 g (3.8 mmol) of sodium carbonate and 0.49 g (3.9 mmol) of benzyl chloride were stirred magnetically under reflux in a 250 mL round-bottom flask for 4 h. The reaction mixture was cooled and filtered followed by the washing of the solids with 50 mL of acetonitrile. The combined filtrates were concentrated by rotary evaporation giving a thick deep yellow-orange oil which was diluted with 50 mL CH₂Cl₂ and stirred vigorously for 5 min with 58 mL saturated aqueous Na₂S₂O₃. The layers were separated and the organic portion was washed twice with 70 mL saturated NaCl. The combined aqueous layers were extracted with CH₂Cl₂, dried over 10 g MgSO₄ and concentrated by rotary evaporation. The residue was purified by column chromatography on alumina with gradient elution using 2:3, 3:2, 4:1 ethyl acetate/hexane mixture then 100% ethyl acetate. Eluting solvent was evaporated to dryness on a rotary evaporator followed by drying at 65° C. at 0.5 mm for 3 days to give a solidifying yellow oil 1.19 g (50%) as pure product.

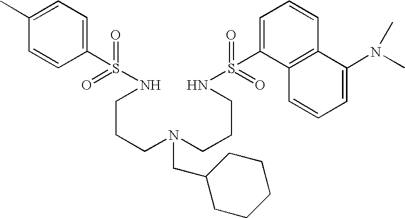

N-(3-(5-dimethylamino-1-napthalenesulfonamidopropyl)-N-(3-p-toluenesulfonamidopropyl) methylcyclohexane

N-[3-(5-dimethylamino-1-napthalenesulfonamidopropyl)]-N-(3-p-toluenesulfonamido-propyl) amine (0.25 g, 0.41 mmol),(0.66 mmol) sodium iodide, 0.40 g (3.8 mmol) sodium carbonate and 0.38 g (2.1 mmol) bromomethyl cyclohexane was stirred magnetically and refluxed in a 250 mL round bottom flask for 4 hr. The reaction mixture was cooled and filtered followed by the washing of the solids with 50 mL of acetonitrile. The combined filtrates were concentrated by rotary evaporation giving a thick deep yellow-orange oil which was diluted with 50 mL CH₂Cl₂ and stirred vigorously for 5 min with 58 mL saturated aqueous Na₂S₂O₃. The layers were separated and the organic portion was washed twice with 70 mL saturated NaCl. The combined aqueous layers were extracted with CH₂Cl₂, dried over 10 g MgSO₄ and concentrated by rotary evaporation. The residue was purified by column chromatography on alumina with gradient elution using 2:3, 3:2, 4:1 ethyl acetate/hexane mixture the 100% ethyl acetate. Eluting solvent was evaporated to dryness on a rotary evaporator followed by drying at 65° C. at 0.5 mm for 3 days to give a solidifying yellow oil 0.25 g (19%) as pure product.

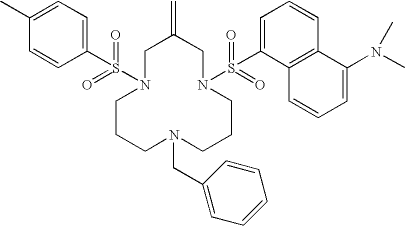

9-Benzyl-3-methylene-1-(5-dimethylamino-1-naphthalenesulfonyl)-5-p-toluenesulfonyl-1,5,9-triazacyclododecane (KKD-015)

In a 250 mL three-necked, round-bottomed flask equipped with a rubber septum, a thermometer, a condenser and a gas inlet, 0.13 g of a 60% (w/w) slurry of sodium hydride in mineral oil (0.078 g NaH, 3.3 mmol) was washed under N₂ with hexane (2×10 mL). With stirring, 40 mL of anhydrous DMF was added, and the resulting mixture was heated to 75° C. N-Benzyl-N-dimethylaminonaphthalenesulfonamidpropyl-N′-p-toluenesulfonamidopropyl amine hydrochloride (0.5 g, 0.8 mmol) solution in 5 mL anhydrous DMF was added. A solution of 0.13 g (0.93 mmol) of 3-chloro-2-chloromethyl-1-propene in 20 mL of DMF was added over a period of 6 h by means of a syringe pump. Upon completion of the addition, stirring at 60° C. under N₂ was continued for 12 h. The reaction mixture was allowed to cool and the solvent was removed on a rotary evaporator. A solution of the residue in 75 mL of CHCl₃ was washed with water (3×50 mL) followed by concentration to dryness to give 1.12 g of the crude product. Partial purification of this product was done by column chromatography on silica gel and eluting with 2:3 ethyl acetate/hexane solvent mixture. The yellow solid that resulted after evaporation of solvents was recrystallized from diethyl ether and hexane to give a final yield of 0.14 g (27%).

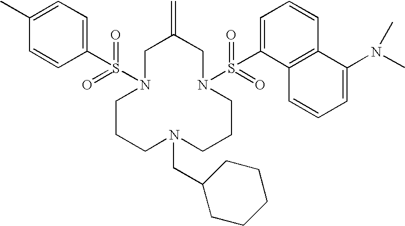

9-methylcyclohexane-3-methylene-1-(5-dimethylamino-1-naphthalenesulfonyl)-5-p-toluenesulfonyl-1, 5,9-triazacyclododecane (KKD-016)

In a 250 mL three-necked, round-bottomed flask equipped with a rubber septum, a thermometer, a condenser and a gas inlet, 0.06 g of a 60% (w/w) slurry of sodium hydride in mineral oil (0.036 g NaH, 1.6 mmol) was washed under N₂ with hexane (2×10 mL). With stirring, 40 mL of anhydrous DMF was added, and the resulting mixture was heated to 75° C. N-methylcyclohexane-N-dimethylaminonaphthalenesulfonamidpropyl-N′-p-toluenesulfon-amidopropyl amine hydrochloride (0.25 g, 0.41 mmol) solution in 5 mL anhydrous DMF was added. A solution of 0.05 g (0.41 mmol) of 3-chloro-2-chloromethyl-1-propene in 20 mL of DMF was added over a period of 6 h by means of a syringe pump. Upon completion of the addition, stirring at 60° C. under N₂ was continued for 12 h. The reaction mixture was allowed to cool and the solvent was removed on a rotary evaporator. A solution of the residue in 75 mL of CHCl₃ was washed with water (3×50 mL) followed by concentration to dryness to give the crude product. Partial purification of this product was done by column chromatography on silica gel and eluting with 3:7 ethyl acetate/hexane solvent mixture. The yellow solid that resulted after evaporation of solvents was recrystallized from CH₂Cl₂ and diethyl ether to give 0.10 g (35%) of yellow viscous oil, which was converted to the HCl salt.

Measurements of CC₅₀, IC₅₀ (CD4) and IC₅₀ (HIV-1) for KKD015 and KKD016 were performed as discussed in the Examples above to give the following results compares to CADA for MT4 cells and SupT1 cells:

TABLE 6

| | MT4 | | | SupT1 | |
|---|---|---|---|---|---|
| Compound | CC₅₀ | IC₅₀ (CD4) | IC₅₀(HIV-1) | CC₅₀ | IC₅₀(CD4) |
| CADA | | 0.35 | 0.52 | | 0.48 |
| KKD015 | >100 | 0.97 | 6.7 | >100 | 1.45 |
| KKD016 | 61.2 | 0.41 | 1.15 | 27.4 | 0.49 |

Those of ordinary skill in the art will appreciate that materials, synthetic methods, assay methods, substituents and structural variations of triaza compounds other than those specifically exemplified herein can be employed in the practice of this invention without resort to undue experimentation. Those of ordinary skill in the art will also appreciate that materials, synthetic methods, assay methods, substituents and structural variations exist and are well-known in the art that are functionally equivalent to the materials, synthetic methods, assay methods, substituents and structural variations specifically exemplified. All such art known equivalents are intended to be encompassed by this invention. All references cited herein are incorporated by reference in their entirety herein.

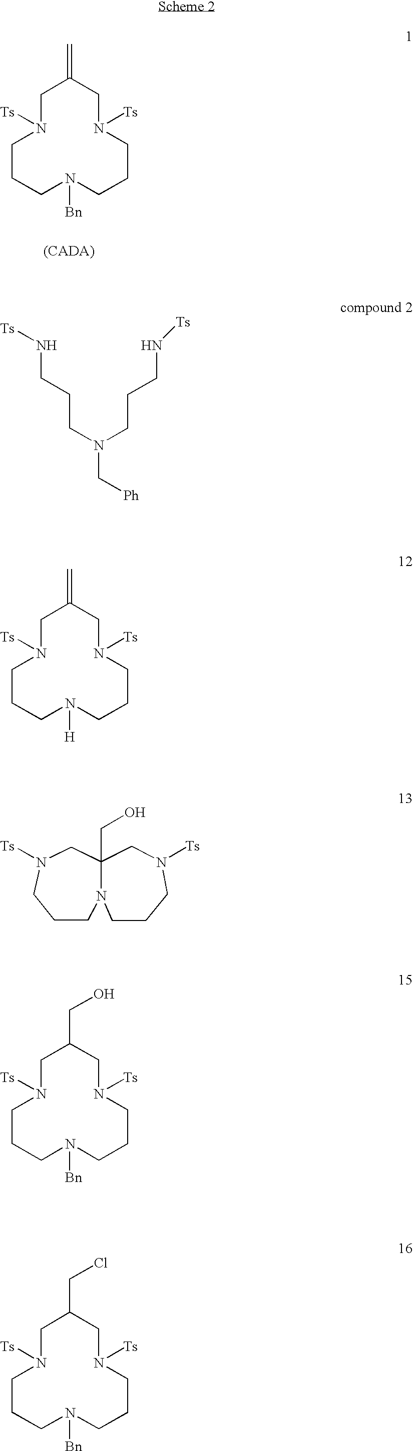

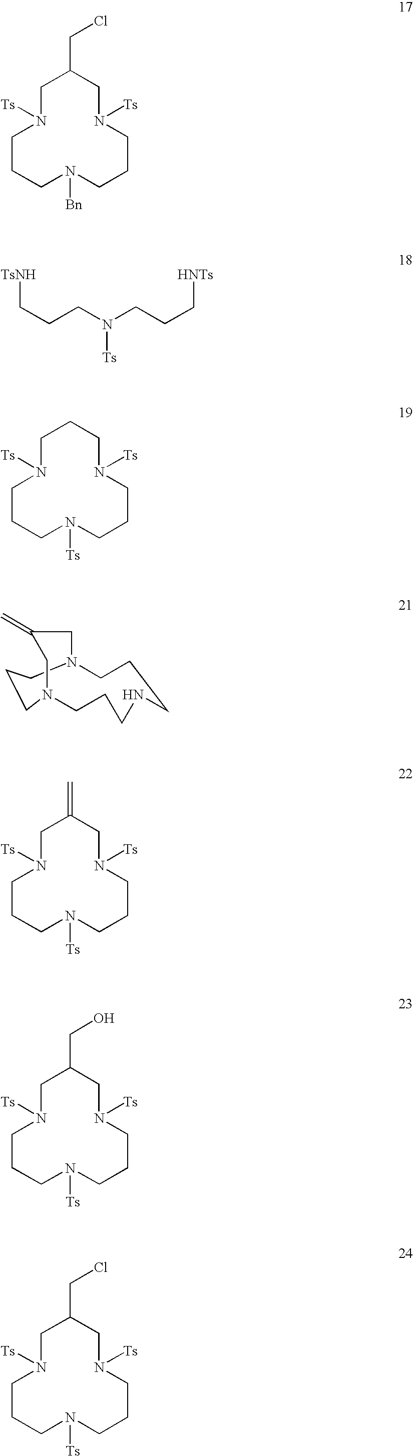

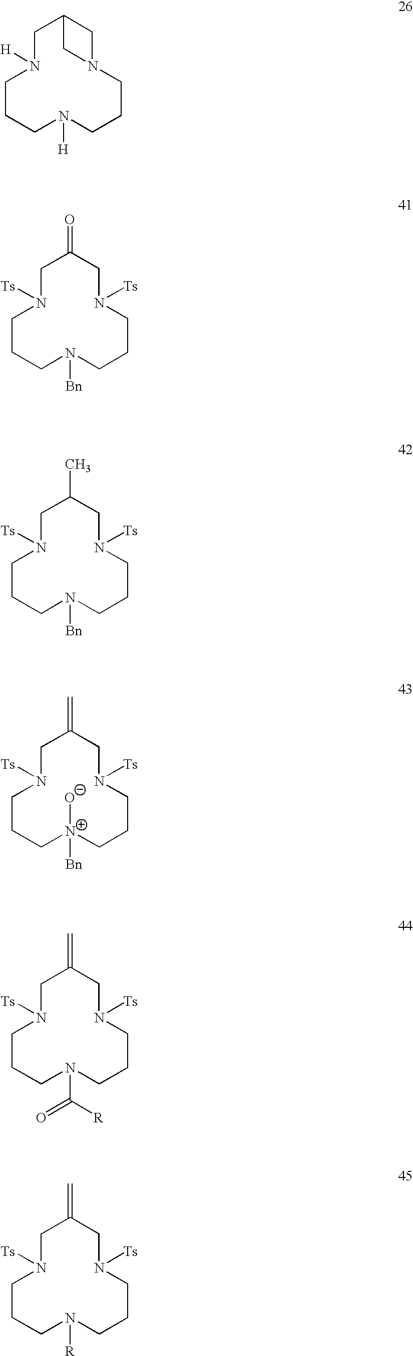

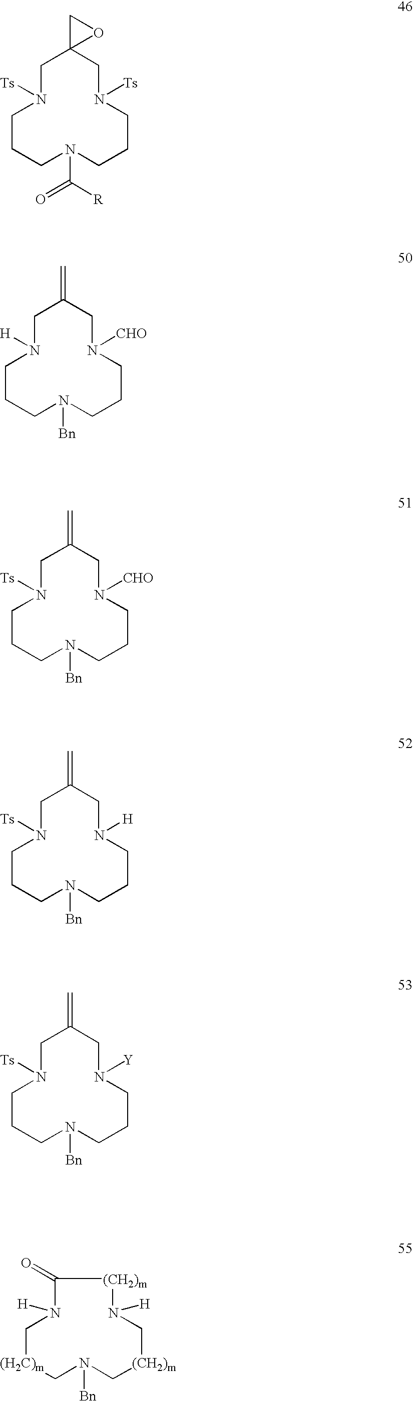

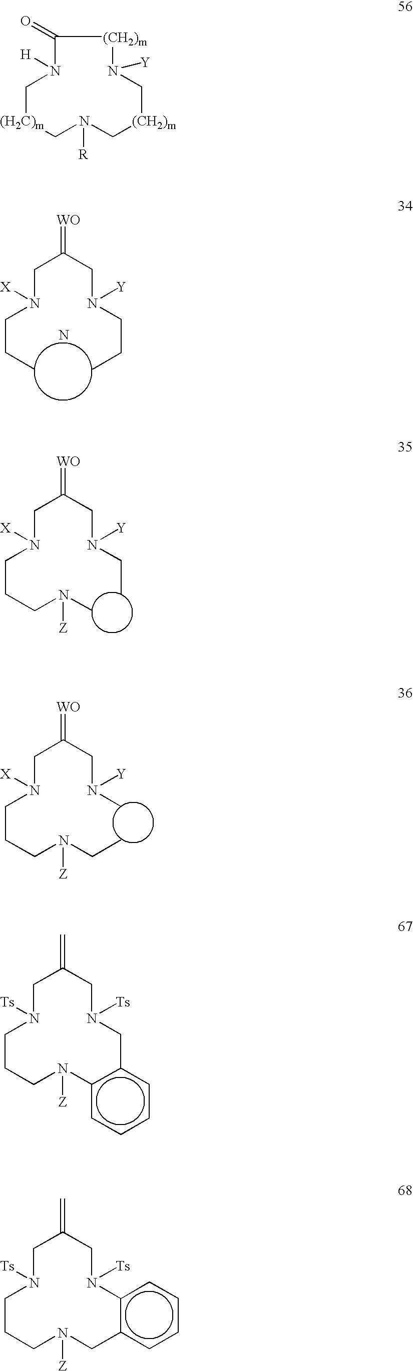

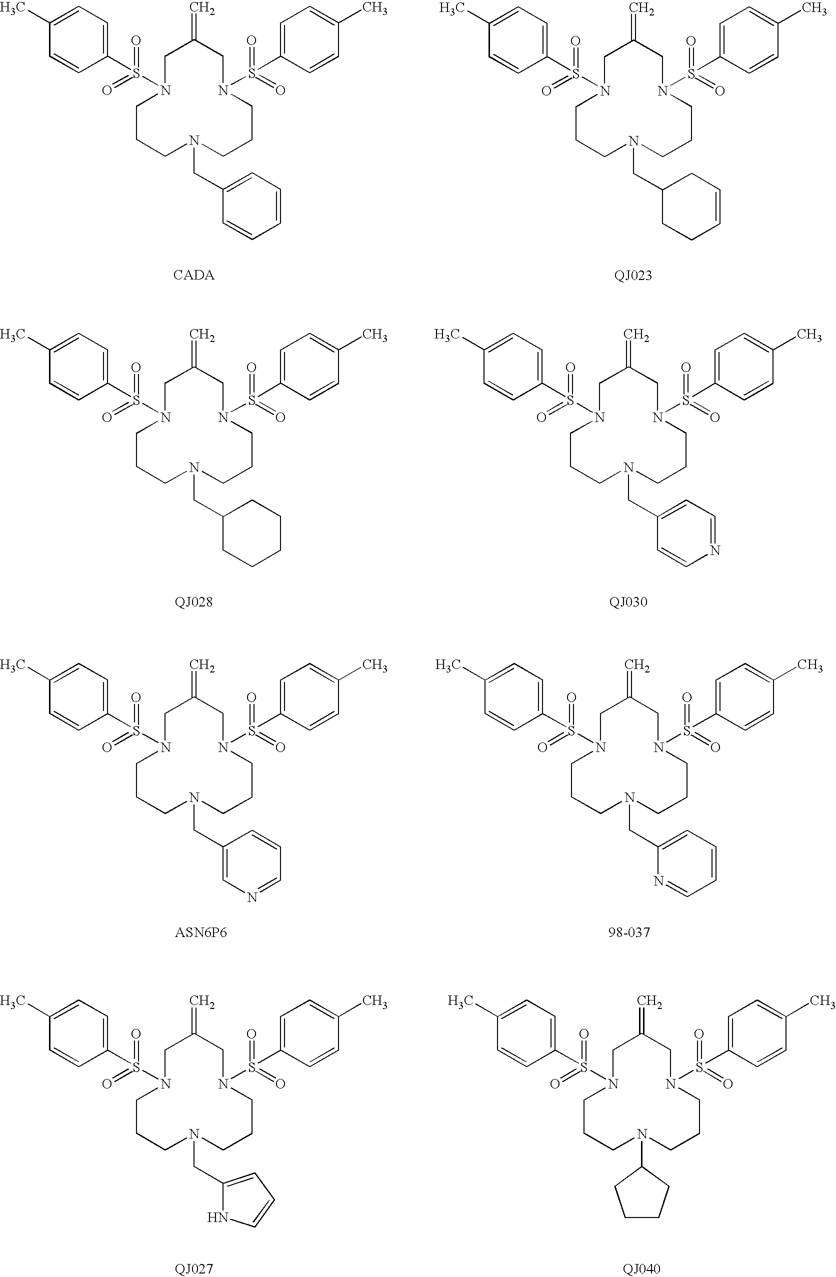

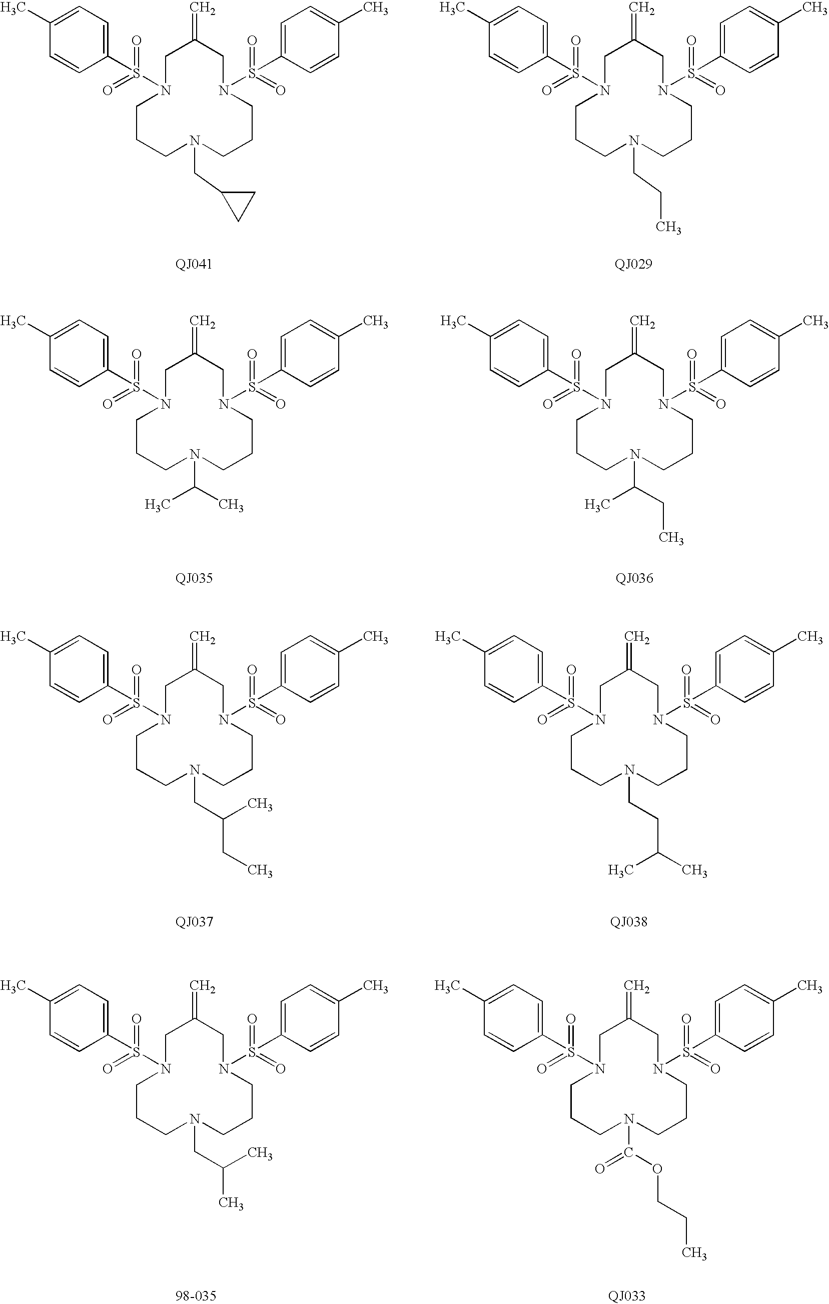

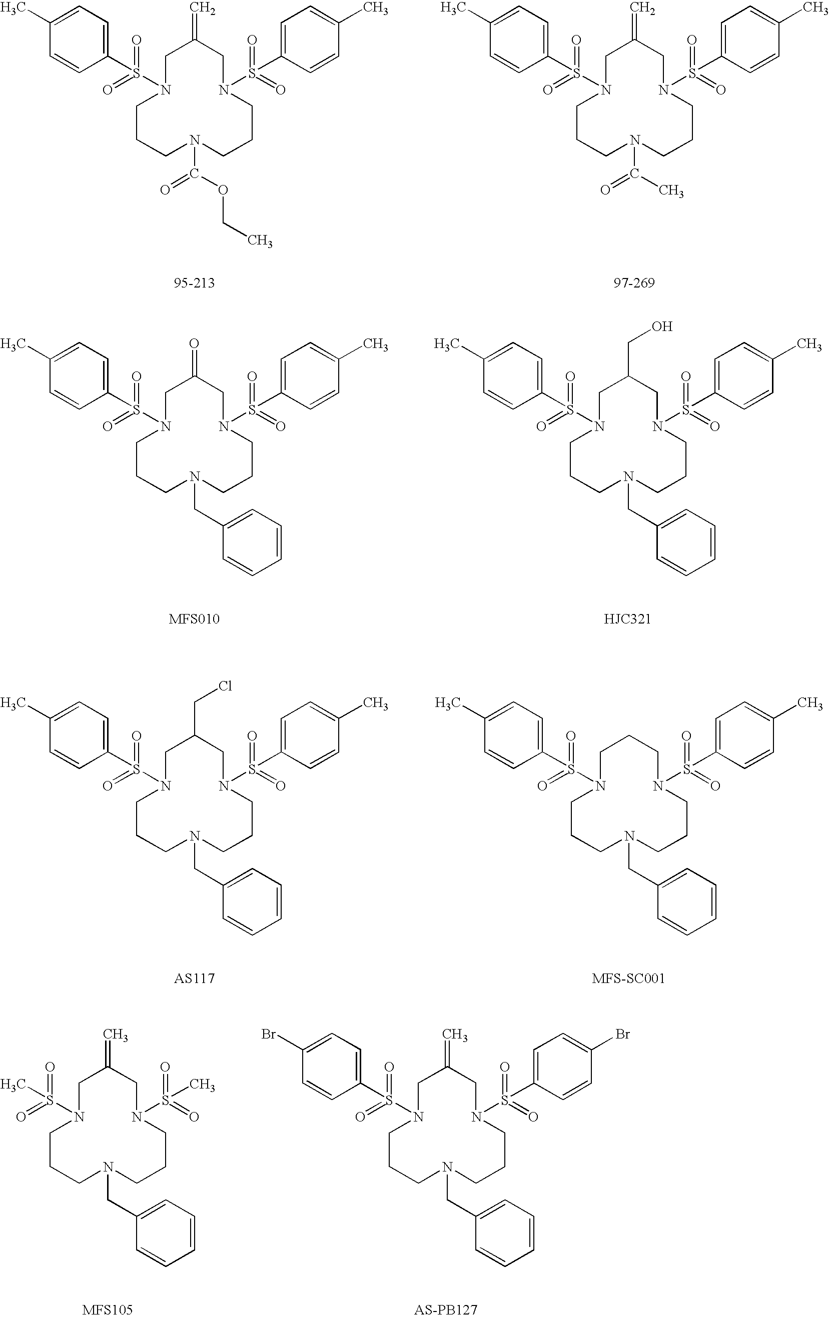

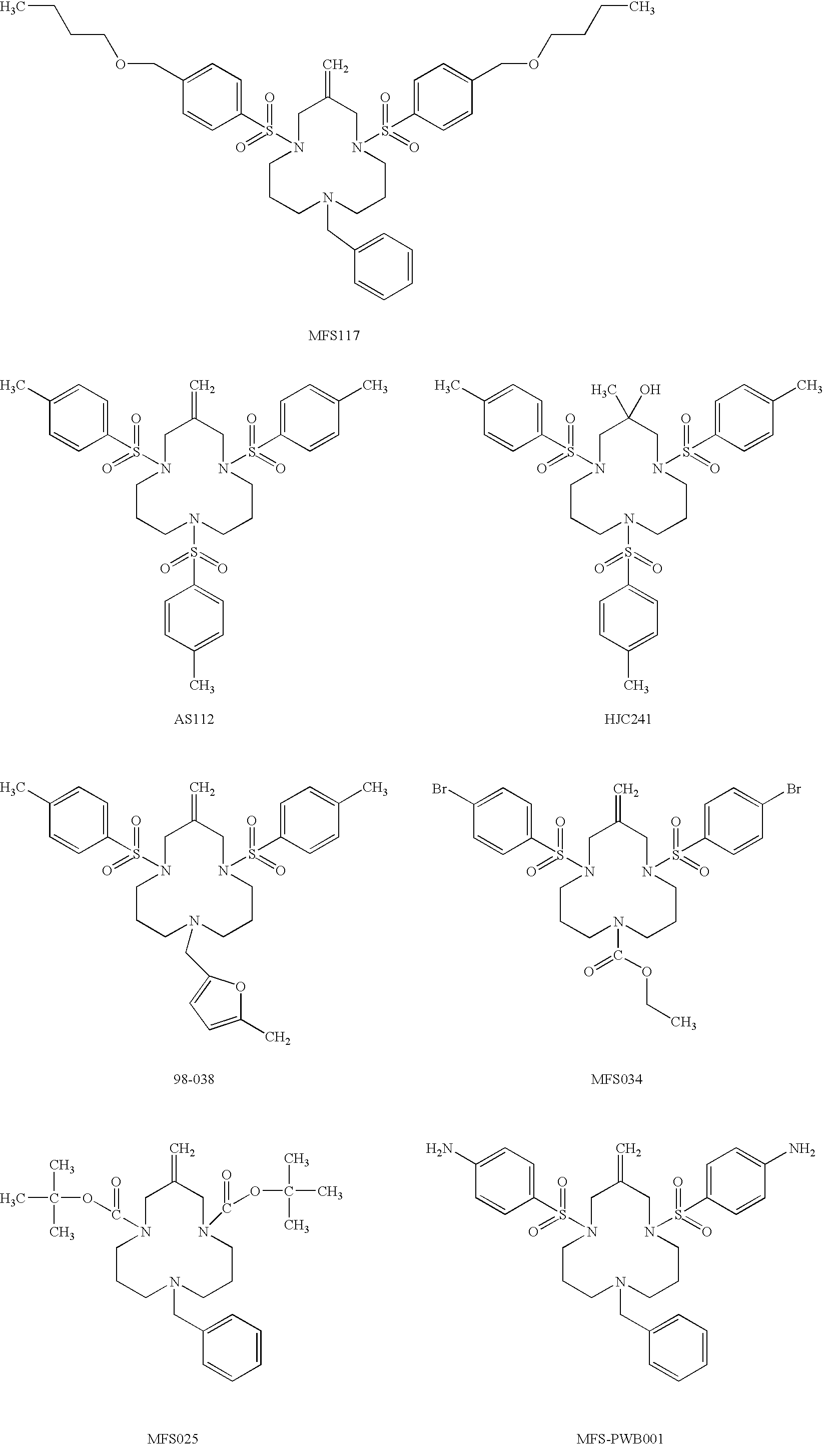

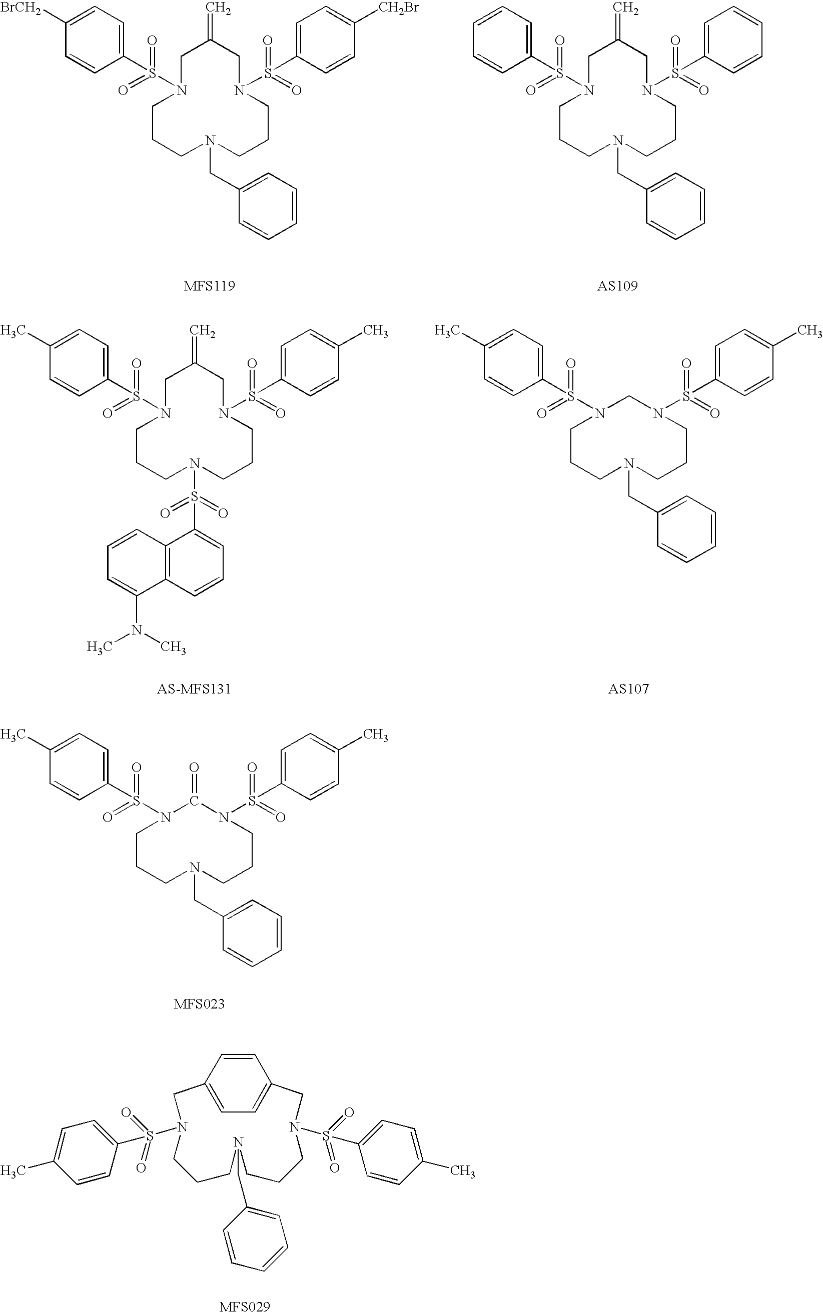

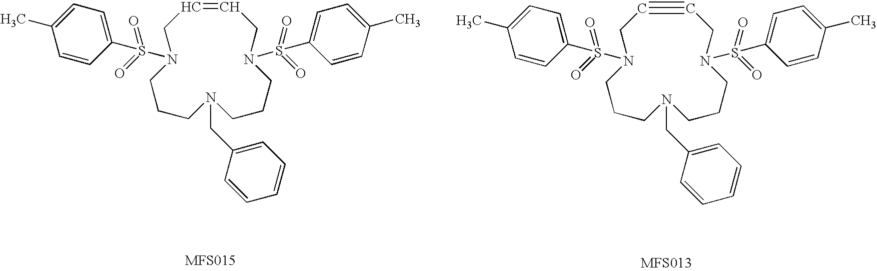

We claim:

1. A method for treating an individual suffering from a pathological conditions which is ameloriated by suppression of CD4+-T-cell-mediated immune response, other than a condition resulting from viral infection, which comprises the step of administering to the individual a therapeutically effective amount of a triaza macrocyclic compound having the formula: 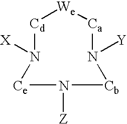
or a pharmaceutically acceptable salt or solvate thereof wherein:
W represents a bridge carbon which is unsubstituted or which is bonded directly or indirectly to one or two polar or non-polar side group substituents selected from the group consisting of double-bonded carbon (═C(H)₂ or ═C(R)₂), double bonded oxygen (═O), hydroxyl, alkyl of about one to 10 carbons, alkenyl of about two to 10 carbons (preferably of 2 to 6 carbon atoms); a substituted alkyl group carrying a charged substituent, such as an —S(R″)₂⁺, an —N(R″)₃⁺, a —PR₃⁺, or an —OSO₃⁻ group, alkoxy of about one to 10 carbons; aryl of about 6 to 12 carbons; halogen, methyl halogen (—CT₃, —CHT₂, or —CH₂T), methylene halide (═CT₂); optionally substituted epoxide (or oxirane); acyl (—CO—R); (—CO₂—R); CH₂OH and hydrogen; where halogen is F, Cl, I or Br; T, independently of other T, is F, Cl, I or Br, but preferably all T are the same halogen; R, independently of other R, is an optionally substituted alky of about one to 10 carbons (preferably of one to 6 carbon atoms), an optionally substituted alkenyl group of about 2 to 10 carbon atoms or an optionally substituted aryl group of about 6 to 12 carbons; and R″ is a hydrogen or an alkyl group having from one to 10 carbon atoms and W may be bonded to one hydrogen and one polar or non-polar group;
X and Y, independently, represent an optionally substituted aryl group (Ar), an optionally substituted alkyl group having from one to 10 carbon atoms, or an optionally substituted alkenyl group having from 2 to 10 carbon atoms attached to the triaza macrocycle through an optional linker group L; where the linker group L can be sulfonyl (—SO₂—), —SO—, —PO—, —PO(OH)—, —PO(H)—, —PO₂(OH)—, —PO₂(H)—, —PO₃(OH)—, carboxy (—OCO—), carbonyl (—CO—), or —(CH₂)ₙ—where n is 1 or 2-; where Ar comprises at least one aromatic homocyclic or heterocyclic ring having from five to seven members; wherein the Ar ring can be substituted with one or more non-hydrogen substituent groups; Ar group substituents are one or more halogens, one or more —CN; one or more —SO3, —SH, —SR or —S—OR groups; one or more trihalomethyl groups; one or more NO, one or more NO₂, one or more NH₂, NHR or N(R)₂ groups; one or more alkyl groups, one or more alkoxy groups, one or more hydroxyl groups, one or more acyl groups (—COH or —CO—R), one or more acid or ester groups (—CO₂H or —CO₂R, respectively), where R, independently of other R, is an alky of about one to 10 carbons or an aryl group of about 7 to 10 carbons and wherein X and Y are not both an alkyl group;
Z represents a hydrogen, or optionally substituted aryl, alkyl or alkenyl groups attached to the triaza macrocycle though a linking group L³, wherein the aryl, alkyl and alkenyl groups and the linking group of Z are as described under X and Y variables above;
C labeled with subscripts a-d represent carbon bridges, preferably alkylene bridges, between nitrogens, these carbon bridges, the length of which is defined by the values of subscripts a-d and e, may all be the same length or may differ in length, each bridge may be composed entirely of saturated alkyl groups, or one or more bridges may contain one or more double or triple bonds between carbons, additionally one or more bridge carbons can be optionally substituted with one or more polar groups, and aromatic rings, non-aromatic rings or both may be fused to one or more of the carbon atom bridges; and
a and d, independently, represent a number from zero to 10; b and c, independently, represent a number from one to 10; and e represents a number from zero to three.

2. The method of claim 1 wherein e is 1 and W is double-bonded carbon (═C(H)₂ or ═C(R)₂), a double bonded oxygen (═O), a methylene halide, or a carbon bonded to one or two groups selected from hydrogen, hydroxyl, alkyl groups of about one to 10 carbons, alkenyl groups of about two to 10 carbons, a substituted alkyl group carrying a charged substituent, alkoxy groups of about one to 10 carbons; aryl groups of about 6 to 12 carbons; halogens, methyl, an optionally substituted epoxide (or oxirane); acyl (—CO—R); (—CO₂—R); and CH₂OH; where the halogen is F, Cl, I or Br; and R independently of other R, is an optionally substituted alky of about one to 10 carbons, an optionally substituted alkenyl group of about 2 to 10 carbon atoms or an optionally substituted aryl group of about 6 to 12 carbons and R″ is a hydrogen or an alkyl group having from one to 10 carbon atoms.

3. The method of claim 2 wherein W is >C═C(H)₂, >C═C(R)₂, or >C(T)₂.

4. The method of claim 1 wherein and Y independently represent an optionally substituted aryl group attached to the triaza macrocycle through an optional linker group L; where the linker group L can be —SO₂—, —SO—, —PO—, —PO(OH)—, —PO(H)—, —PO₂(OH)—, —PO₂(H)—, —PO₃(OH)—, —CO—, —CO—, or alkyl.

5. The method of claim 4 wherein L is —SO₂—.

6. The method of claim 5 wherein X and Y are selected from tosyl groups or dansyl groups.

7. The method of claim 1 wherein Z is an optionally substituted aryl, alkyl or alkenyl group attached to the triaza macrocycle though a linking group L³ selected from the groups consisting of —SO₂—, —SO—, —PO—, —PO(OH)—, —PO(H)—, —PO₂(OH)—, —PO₂(H)—, —PO₃(OH)—, —OCO—, —CO—, and alkyl.

8. The method of claim 7 wherein L³ is an alkyl, —CO—or —OCO—group.

9. The method of claim 7 wherein Z is a benzyl groups, a methylene cyclohexane group, or a methylene cyclohexene group.

10. The method of claim 1 wherein a, d and e are all 1 and b and c are 3.

11. The method of claim 1 wherein the pathological condition is an autoimmune disorder or a chronic inflammatory disease.

12. The method of claim 1 wherein the pathological condition is graft-versus host disease or transplant rejection.

13. The method of claim 1 wherein the pathologic condition is rheumatoid arthritis, type I-diabetes mellitus, autoimmune demyelinating diseases such as multiple sclerosis, inflammatory bowel disease syndrome, psoriasis, discoid lupus erythematosus, systemic lupus erythematosus (SLE),adult respiratory distress syndrome, cardiovascular atherosclerosis, leukocytosis, or asthma.

14. A method for downregulating CD4 expression on T cells by exposing the T cells to an amount of a triaza compound of formula: 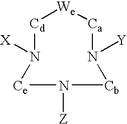
or a pharmaceutically acceptable salt or solvate thereof that is effective for downregulating expression of CD4
wherein:
W represents a bridge carbon which is unsubstituted or which is bonded directly or indirectly to one or two polar or non-polar side group substituents selected from the group consisting of double-bonded carbon (═C(H)₂ or ═C(R)₂), double bonded oxygen (═O), hydroxyl, alkyl of about one to 10 carbons alkenyl of about two to 10 carbons; a substituted alkyl group carrying a charged substituent, such as an —S(R″)₂⁺, an —N(R″)₃⁺, a —PR₃⁺, or an —OSO₃^{—}group, alkoxy of about one to 10 carbons; aryl of about 6 to 12 carbons; halogen, methyl halogen(—CT₃, —CHT₂, or —CH₂T), methylene halide (═CT₂); optionally substituted epoxide (or oxirane); acyl (—CO—R); (—CO₂—R); CH₂OH and hydrogen; where halogen is F, Cl, I or Br; T, independently of other T, is F, Cl, I or Br, but preferably all T are the same halogen; R, independently of other R, is an optionally substituted alky of about one to 10 carbons, an optionally substituted alkenyl group of about 2 to 10 carbon atoms or an optionally substituted aryl group of about 6 to 12 carbons and R″ is a hydrogen or an alkyl group having from one to 10 carbon atoms and W may be bonded to one hydrogen and one polar or non-polar group;
X and Y independently represent an optionally substituted aryl group (Ar), an optionally substituted alkyl group having from one to 10 carbon atoms, or an optionally substituted alkenyl group having from 2 to 10 carbon atoms attached to the triaza macrocycle through an optional linker group L; where the linker group L can be sulfonyl (—SO₂—), —SO—, —PO—, —PO(OH)—, —PO(H)—, —PO₂(OH)—, —PO₂(H)—, —PO₃(OH)—, carboxy (—OCO—), carbonyl (—CO—), or —(CH₂)ₙ—where n is 1 or 2-; where Ar comprises at least one aromatic homocyclic or heterocyclic ring having from five to seven members; wherein the Ar ring can be substituted with one or more non-hydrogen substituent groups; Ar group substituents include one or more halogens, one or more —CN; one or more —SO₃, —SH, —SR or —S—OR groups; one or more trihalomethyl groups; one or more NO, one or more NO₂, one or more NH₂, NHR or N(R)₂ groups; one or more alkyl groups, one or more alkoxy groups, one or more hydroxyl groups, one or more acyl groups, one or more acid or ester groups (—CO₂H or —CO₂R, respectively), where R, independently of other R, is an alky of about one to 10 carbons or an aryl group of about 7 to 10 carbons and wherein X and Y are not both an alkyl group;
Z represents a hydrogen, or optionally substituted aryl, alkyl or alkenyl groups attached to the triaza macrocycle though a linking group L³, wherein the aryl, alkyl and alkenyl groups and the linking group of Z are as described under X and Y variables above;
C labeled with subscripts a-d represent carbon bridges, preferably alkylene bridges, between nitrogens, these carbon bridges, the length of which is defined by the values of subscripts a-d and e, may all be the same length or may differ in length, each bridge may be composed entirely of saturated alkyl groups, or one or more bridges may contain one or more double or triple bonds between carbons, additionally one or more bridge carbons can be optionally substituted with one or more polar groups, for example, halogens or hydroxy groups, and additionally aromatic, non-aromatic rings or both may be fused to one or more of the carbon atom bridges; and
a and d, independently, represent a number from zero to 10; b and c, independently, represent a number from one to 10; and e represents a number from zero to three.

15. The method of claim 14 wherein e is 1 and W is double-bonded carbon (═C(H)₂ or ═C(R)₂), a double bonded oxygen (═O), a methylene halide, or a carbon bonded to one or two groups selected from hydrogen, hydroxyl, alkyl groups of about one to 10 carbons, alkenyl groups of about two to 10 carbons, a substituted alkyl group carrying a charged substituent, alkoxy groups of about one to 10 carbons; aryl groups of about 6 to 12 carbons; halogens, methyl, an optionally substituted epoxide (or oxirane); acyl (—CO—R); (—CO₂—R); and CH₂OH; where the halogen is F, Cl, I or Br; and R independently of other R, is an optionally substituted alky of about one to 10 carbons, an optionally substituted alkenyl group of about 2 to 10 carbon atoms or an optionally substituted aryl group of about 6 to 12 carbons and R″ is a hydrogen or an alkyl group having from one to 10 carbon atoms.

16. The method of claim 15 wherein W is ═C(H)₂ or ═C(R)₂, or >C(T)₂.

17. The method of claim 14 wherein X and Y independently represent an optionally substituted aryl group attached to the triaza macrocycle through an optional linker group L; where the linker group L can be —SO₂—, —SO—, —PO—, —PO(OH)—, —PO(H)—, —PO₂(OH)—, —PO₂(H)—, —PO₃—(OH)—, —OCO—, —CO—, or alkyl.

18. The method of claim 17 wherein L is —SO₂—.

19. The method of claim 18 wherein X and Y are selected from tosyl groups or dansyl groups.

20. The method of claim 14 wherein Z is an optionally substituted aryl, alkyl or alkenyl group attached to the triaza macrocycle though a linking group L³ selected from the groups consisting of —SO₂—, —SO—, —PO—, —PO(OH)—, —PO(H)—, —PO₂(OH)—, —PO₂(H)—, —PO₃(OH)—, —OCO—, —CO—, or alkyl.

21. The method of claim 20 wherein L³ is an alkyl, —CO—or —OCO—group.

22. The method of claim 20 wherein Z is a benzyl groups a methylene cyclohexane group or a methylene cyclohexene group.

23. The method of claim 14 wherein a, d and e are all 1 and b and c are 3.

24. A pharmaceutical composition comprising a pharmaceutically acceptable carrier and a therapeutically effective combined amount of one or more triaza macrocycle compounds of formula: 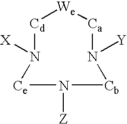
or a pharmaceutically acceptable salt or solvate thereof that is effective for downregulating expression of CD4
wherein:
W represents a bridge carbon which is unsubstituted or which is bonded directly or indirectly to one or two polar or non-polar side group substituents selected from the group consisting of double-bonded carbon, double bonded oxygen, hydroxyl, alkyl of about one to 10 carbons alkenyl of about two to 10 carbons; a substituted alkyl group carrying a charged substituent, such as an —S(R″)₂⁺, an —N(R″)₃⁺, a —PR₃⁺, or an —OSO₃⁻group, alkoxy of about one to 10 carbons; aryl of about 6 to 12 carbons; halogen, methyl halogen(—CT₃, —CHT₂, or —CH₂T), methylene halide (═CT₂); optionally substituted epoxide (or oxirane); acyl (—CO—R); (—CO₂—R); CH₂OH and hydrogen; where halogen is F, Cl, I or Br; T, independently of other T, is F, Cl, I or Br, but preferably all T are the same halogen; R, independently of other R, is an optionally substituted alky of about one to 10 carbons, an optionally substituted alkenyl group of about 2 to 10 carbon atoms or an optionally substituted aryl group of about 6 to 12 carbons and R″ is a hydrogen or an alkyl group having from one to 10 carbon atoms and W may be bonded to one hydrogen and one polar or non-polar group;
X and Y independently represent an optionally substituted aryl group (Ar), an optionally substituted alkyl group having from one to 10 carbon atoms, or an optionally substituted alkenyl group having from 2 to 10 carbon atoms attached to the triaza macrocycle through an optional linker group L; where the linker group L can be sulfonyl (—SO₂—), —SO—, —PO—, —PO(OH)—, —PO(H)—, —PO₂(OH)—, —PO₂(H)—, —PO₃(OH)—, carboxy (—OCO—), carbonyl (—CO—), or —(CH₂)ₙ—where n is 1 or 2-; where Ar comprises at least one aromatic homocyclic or heterocyclic ring having from five to seven members; wherein the Ar ring can be substituted with one or more non-hydrogen substituent groups; Ar group substituents include one or more halogens, one or more —CN; one or more —SO₃, —SH, —SR or —S—OR groups; one or more trihalomethyl groups; one or more NO, one or more NO₂, one or more NH₂, NHR or N(R)₂ groups; one or more alkyl groups, one or more alkoxy groups, one or more hydroxyl groups, one or more acyl groups (—COH or —CO—R), one or more acid or ester groups (—CO₂H or —CO₂R, respectively), where and R, independently of other R, is an alky of about one to 10 carbons or an aryl group of about 7 to 10 carbons and wherein X and Y are not both an alkyl group;
Z represents a hydrogen, or optionally substituted aryl, alkyl or alkenyl groups attached to the triaza macrocycle though a linking group L³, wherein the aryl, alkyl and alkenyl groups and the linking group of Z are as described under X and Y variables above;
C labeled with subscripts a-d represent carbon bridges, preferably alkylene bridges, between nitrogens, these carbon bridges, the length of which is defined by the values of subscripts a-d and e, may all be the same length or may differ in length, each bridges may be composed entirely of saturated alkyl groups, or one or more bridges may contain one or more double or triple bonds between carbons, additionally one or more bridge carbons can be optionally substituted with one or more polar groups, and additionally aromatic, non-aromatic rings or both may be fused to one or more of the carbon atom bridges; and
a and d, independently, represent a number from zero to 10; b and c, independently, represent a number from one to 10; and e represents a number from zero to three.

25. A triaza macrocylic compound of formula: 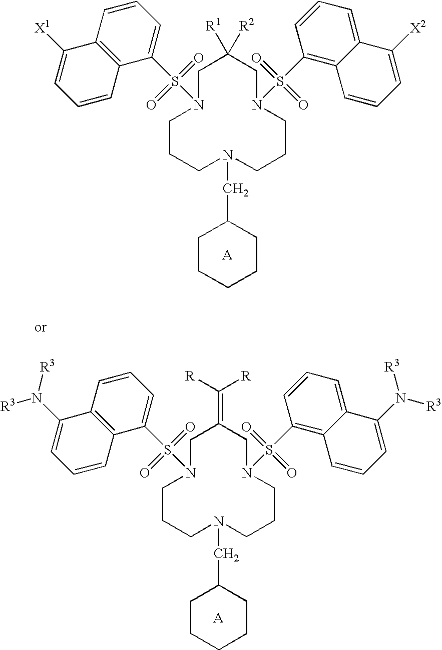
or a pharmaceutically acceptable salt or solvate thereof wherein:
X¹ and X² independently can be a charged, polar or non-polar substituent;
the A ring is an optionally substituted phenyl ring, an optionally substituted cyclohexane ring or an optionally substituted cyclohexene ring;
R¹ and R² represent substituents on the central carbon of one of the carbon bridges which independently, can be a hydrogen, hydroxyl, halogen, an optionally substituted alkyl group having one to 10 carbon atoms, an optionally substituted alkenyl group having 2 to 10 carbon atoms; optionally substituted alkoxy of about one to 10 carbons; methyl halogen(—CT₃,—CHT₂, or —CH₂T); epoxide (or oxirane); acyl (—CO—R); ester (—CO₂—R); CH₂OH and hydrogen; or R¹ and R² together can represent a double-bonded carbon which in turn is bonded to one or two hydrogens and/or R′groups (i.e., ═CH₂, ═CRH, or ═C(R)₂), methylene halide (═CT₂);or a double bonded oxygen (═O)., where halogen is F, Cl, I or Br; T, independently of other T, is F, Cl, I or Br, and
R and R³, independently of other R and R³, is an optionally substituted alkyl, ether or thioether of about one to 10 carbons or an aryl group of about 7 to 10 carbons and wherein the R groups are optionally substituted and two R in the sarne group can form a cyclic moiety.

26. The compound of claim 25 wherein X¹ and X² are alkyl amines.

27. The compound of claim 26 wherein the A ring is an optionally substituted phenyl ring.

28. A pharmaceutical composition comprising a pharmaceutically acceptable carrier and one or more compounds of claim 25 in a combined amount that is therapeutically effective.
